(19) United States
(12) Patent Application Publication
(10) Pub. No.: US 2015/0314102 A1
(43) Pub. Date: Nov. 5, 2015

(54) MULTIMODAL MEDICAL TREATMENT OPTIMIZATION SYSTEM AND METHOD

(71) Applicant: Leon I. Rosenberg, Cherry Hill, NJ (US)

(72) Inventor: Leon I. Rosenberg, Cherry Hill, NJ (US)

(21) Appl. No.: 14/704,723

(22) Filed: May 5, 2015

Related U.S. Application Data
(60) Provisional application No. 61/988,854, filed on May 5, 2014.

(51) Int. Cl. A61M 21/00; A61M 21/02; A61N 2/00; G06F 3/16

(52) U.S. Cl. CPC A61M 21/00; G06F 3/165; A61M 21/02; A61N 2/002; A61M 15/0001

(57) ABSTRACT

A system and method for multimodal medical treatment optimization. The system and method involve (1) providing a patient with a primary medical treatment via a first delivery system, (2) providing a patient with an adjunctive medical treatment via a second delivery system integrated with the first delivery system, and (3) adjusting at least one of the primary medical treatment and the adjunctive medical treatment as a result of the patient's response to at least one of the primary medical treatment and the adjunctive medical treatment to promote adherence/compliance, efficacy and/or other factors associated with a patient's treatment regimen.

### CROSS REFERENCE TO RELATED APPLICATION

This application claims the benefit of U.S. Provisional Application No. 61/988,854, filed May 5, 2014, entitled “Multi Modal Treatment Optimization,” the disclosure of which is incorporated herein in its entirety by reference.

### BACKGROUND OF THE INVENTION

The present invention relates in general to medical treatment systems and methods and, in particular, to multimodal medical treatment optimization systems and methods.

Medical expenses continue to rise due to better treatments costing more money. Private industry is one of the driving forces for improved health care, and the government is involved in regulating all new medications and treatments. Significant research and development funds are earmarked to continue to develop new and improved methods, treatments and techniques.

At the same time, there are many time-proven, cost-effective stress management techniques and other holistic techniques, in and of themselves free of charge, that could augment the benefit of the newer medications and treatments. Both improved adherence to all types of treatments and the combination of treatments can contribute to improved health care and improved health.

The National Association of Chain Drug Stores (NACDS) provided funding for the pamphlet entitled The Role of Medication Adherence in the U.S. Healthcare System, June 2013, that was prepared by Avalere Health (hereinafter “Avalere pamphlet”). The conclusions of the Avalere pamphlet were as follows: “Prescription drugs, when used appropriately, are often the most cost-effective component of the healthcare delivery system. Yet while the clinical and economic benefits to patients from the proper use of prescribed medications are well known, ensuring appropriate medication adherence continues to be difficult within the U.S. healthcare system.”

One of the recent foci of the government as a way to improve the health of the population has been medication adherence. As a result of government oversight, adherence has garnered the attention of pharmacies (the distributors of medication who profit from sales), pharmaceutical manufacturers (the manufacturers of medication who profit from sales), and the insurance companies (who profit from decreased costs of hospitalization). Adherence is defined in the Avalere pamphlet as the “active, voluntary, and collaborative involvement of the patient in a mutually acceptable course of behavior to produce a therapeutic result” (Delamater 2006; Meichenbaum and Turk 1987, cited in Avalere pamphlet). Adherence may be more simply defined as taking medication as directed for as long as directed. In any case, adherence is easier to define than it is to put into action. The lack of adherence to prescribed therapies is termed “medication non-adherence.” Recent research has shown that taking drugs as prescribed lowers total healthcare costs by an amount that exceeds the costs of the drugs themselves. (Roebuck et al. 2011, cited in the Avalere pamphlet.)

Particularly among Medicare patients and individuals with chronic conditions, encouraging the proper use of medicines presents significant challenges. Innovative strategies by insurance providers, physicians, pharmacists and others suggest there are opportunities to incentivize and encourage patients to remain more adherent to prescribed drug regimens. Specific strategies may include lowering copayments, reducing dosing frequency and pharmacist-directed medication therapy management. To improve health outcomes and save money, those who influence healthcare policy and practice should support measures that encourage greater engagement and education with patients to demonstrate the importance of proper medication use.

Other information from the Avalere pamphlet noted that 81 percent of adults aged 65 and older take one or more prescription drugs a day, and 29 percent take five or more medications on a regular basis (Qato et al. 2008). However, approximately one-half of patients in the United States do not take their medications as prescribed (Sabate 2003).

A meta-analysis assessing the results of numerous studies found that relative to patients with high levels of adherence, the risk of poor clinical outcomes-including hospitalization, re-hospitalization, and premature death-among non-adherent patients is 5.4 times as high (compared to those who are adherent) among those with hypertension, 2.8 times as high (compared to those who are adherent) among those with dyslipidemia, and 1.5 times as high (compared to those who are adherent) among those with heart disease (Gwadry-Sridhar et al. 2009, cited in the Avalere pamphlet). Another study found that patients who do not adhere to their high cholesterol medications have a 26 percent greater likelihood of a cardiovascular-related hospitalization, as compared to patients who adhere to their drug regimens (Pittman et al. 2011, cited in the Avalere pamphlet).

A subsequent study of Medicare patients diagnosed with diabetes found that patients who were adherent to cardiovascular drugs as part of their treatment therapy had lower total healthcare costs within the Medicare system over three years, with savings from medical costs outweighing additional costs from greater prescription drug use (Stuart et al. 2011, cited in the Avalere pamphlet).

A report conducted by the New England Healthcare Institute (NEHI), <http://www.nehi.net/bendthecurve/sup/documents/Medication_Adherence_Brief. pdf>, found that not taking medications as prescribed leads to poorer health, more frequent hospitalization, a higher risk of death and as much as $290 billion annually in increased medical costs. Anywhere from one-third to one-half of patients in the U.S. do not take their medications as instructed. Not taking the medication on time, in the proper doses, or at all are just some examples of poor patient medication adherence. Reasons for not following the proper medication regimen include: unpleasant side effects, confusion, forgetfulness, language barriers, and feeling “too good” to need medicine. Those with chronic conditions such as diabetes and high blood pressure and psychiatric illnesses are among the groups that are less likely to follow their medication regimen.

The recent NEHI report is an update of their 2001 report. The more recent report is based on seven systematic reviews of the medical literature plus interviews with 16 health care organizations, insurers, drug makers and technology companies.

According to the NEHI report, NEHI executive director Valerie Fleishman told the Boston Globe in an interview: “If physicians and other care providers are reimbursed for better health outcomes, we believe that will go a long way toward driving adherence because providers will have incentives to invest in the time and resources and counseling and technology and other tools that are really needed to educate patients and in some cases to change their behavior and to really move the needle on adherence.”

There are many wireless health tools and services that can help people follow their medication regimens. Vitality, Inc. made its GlowCaps product commercially available for $99 on Amazon.com. Verizon Wireless has also informed its subscribers that Vocel Inc.'s “The Pill Phone” application is available for download. There are also a number of iPhone apps for medication adherence guidance, including The Pill Phone, myPillBox, and more. Healthcare providers are also contemplating sending medication reminders via secure text messaging as well.

Further, in the not too distant future, sensor technology combined with intelligent medicine technology from Proteus Biomedical, Inc. could serve as the ultimate solution to medication adherence issues. Proteus' Raisin technology can track whether and when a patient takes their medication. Proteus' technology can be embedded in the actual pill, just about any pill, the company claims. Proteus envisions a patient's doctor, nurse or even a relative will be able to confirm that the patient took the medicine. They will also be able to track the patient's respiration, heart rate and body temperature from their mobile phones. Another company, Al Cure, urges facial recognition technology to assure adherence.

There are many aspects to successful healing in addition to medication adherence. These include the following: the placebo response; synergistic drug-drug interactions; complementary and alternative approaches; stress management techniques; psychotherapeutic techniques and others that have efficacy as monotherapy and theoretically could be beneficial for healing rather than relying on medication adherence alone. There are very few studies of the efficacy of medication in combination with these other approaches as will be discussed below.

While adherence has been defined as the “active, voluntary, and collaborative involvement of the patient in a mutually acceptable course of behavior to produce a therapeutic result,” and that definition undoubtedly has been primarily if not solely used to discuss medication adherence, the way it is written in no way demands that it should be restricted to the use of medication only. The same definition could apply both individually to the complementary and alternative approaches, stress management techniques, psychotherapeutic techniques and other procedures, techniques, rating scales, diagnostic tools, etc., noted above as well as to medication(s) in combination therewith.

The biopsychosocial model of treatment, one of the standard theories for the understanding of illness(es) and treatment in medicine was developed by George Engel in 1977 in opposition to the increased reliance among physicians solely on the biomedical model (Engel, George, Science, volume 196, number 4286, Apr. 8, 1977). The biopsychosocial model of treatment gradually became a model for understanding the cause of illnesses and the treatment of both medical and psychiatric patients (Engel, George, American Journal of Psychiatry 1980; 137:535-544).

Understanding the biopsychosocial model of treatment of psychiatric patients includes the following: understanding the biological factors that affect their illness(es), diagnosing their biological dysfunction(s), and treating the biological component of their illness(es) and all other aspects of their illness(es) through biological means; understanding the psychological factors that affect their illness(es); diagnosing the psychological dysfunction(s) and treating the psychological component of their illness(es) and all aspects of their illness(es) through psychological means; understanding the social factors that affect their illness(es); and diagnosing and treating the social component of their illness(es) and all aspects of their illness(es) through social means.

The biopsychosocial model of treatment of patients suggests that a multi-modal approach is required for the successful treatment of psychiatric patients. Although the biomedical model of treatment continues to dominate with some physicians and with the manufacturers of somatic treatments and medications, the biopsychosocial model of treatment of patients was considered to be a valuable way to approach both psychiatric and medical patients as well.

Since many if not most medical patients with chronic illnesses have depression and anxiety associated with their chronic illnesses, the multi-modal approach of the biopsychosocial model of treatment of patients is often required for the successful treatment of patients with chronic illnesses. Thus, the multi-modal approach of the biopsychosocial model of treatment applies to most medical patients with chronic illnesses as well.

According to Wikipedia, circa April 2014, the biopsychosocial model includes the following (with footnotes omitted):

“The biopsychosocial model of health is based in part on social cognitive theory. The biopsychosocial model implies that treatment of disease processes, for example type two diabetes and cancer, requires that the health care team address biological, psychological and social influences upon a patient's functioning. In a philosophical sense, the biopsychosocial model states that the workings of the body can affect the mind, and the workings of the mind can affect the body. This means both a direct interaction between mind and body as well as indirect effects through intermediate factors. The biopsychosocial model presumes that it is important to handle the three together as a growing body of empirical literature suggests that patient perceptions of health and threat of disease, as well as barriers in a patient's social or cultural environment, appear to influence the likelihood that a patient will engage in health-promoting or treatment behaviors, such as medication taking, proper diet or nutrition, and engaging in physical activity.

While operating from a biopsychosocial framework requires that more information be gathered during a consultation with a physician, a growing trend in US healthcare (and already well-established in Europe such as in the U.K. & Germany) includes the integration of professional services through integrated disciplinary teams, to provide better care and address the patient's needs at all three levels. As seen, for example in integrated primary care clinics, such as used in the U.K., Germany, U.S. Veteran's Administration, U.S. military and Kaiser Permanente, integrated teams may comprise physicians, nurses, health psychologists, social workers, and other specialties to address all three aspects of the biopsychosocial framework, allowing the physician to focus on predominantly biological mechanisms of the patient's complaints.

Psychosocial factors can cause a biological effect by predisposing the patient to risk factors. An example is that depression by itself may not cause liver problems, but a depressed person may be more likely to have alcohol problems, and therefore liver damage. Perhaps it is this increased risk-taking that leads to an increased likelihood of disease. Most diseases in biopsychosocial discussion are such behaviorally-moderated illnesses, with known high risk factors, or so-called “biopsychosocial illnesses/disorders.” An example of this is type 2 diabetes, which with the growing prevalence of obesity and physical inactivity, is on course to become a worldwide pandemic. For example, approximately 20 million Americans are estimated to have diabetes, with 90% to 95% considered type 2.”

Evidence for the benefit of combined treatments comes from the National Institute of Mental Health (NIMH) sponsored study Multicenter Treatment Approaches to ADHD (MTA), which compared the following: intensive medication management alone; intensive behavioral treatment alone; a combination of both; or routine community care (the control group). The MTA study showed that combined treatments of medication with intensive behavioral treatment was better than either treatment with medication alone or treatment with intensive behavioral treatment alone. More specifically, the MTA primary results were published in December 1999 in the Archives of General Psychiatry. The study indicated that combination treatment and medication management alone were both significantly superior to intensive behavioral treatment alone and to routine community care in reducing ADHD symptoms. The study also showed that these benefits last for as long as 14 months. Post hoc reviews showed that combination treatment was better than medication management alone in other areas of functioning (e.g., anxiety symptoms, academic performance, parent-child relations, and social skills). Combination treatment was consistently superior to routine community care, whereas medication alone or behavioral treatment alone were not.

While in oncology there are many studies demonstrating combination treatments of medications, the implementation of these treatments is able to occur under close supervision in a restricted medical setting. However, there are only a limited number of studies on medication in combination with psychotherapy. The May 2000 edition of the New England Journal of Medicine published the results of a study of 681 outpatients with chronic depression, randomly assigned to nefazodone, CBASP, (cognitive behavioral analysis system of psychotherapy), or both CBASP and nefazodone. The study showed that the overall rate of response (remission or satisfactory response) was 48% in both the nefazodone and the CBASP groups, compared with 72% when both treatments (CBASP and nefazodone) were combined. For the 519 subjects completing the study, the comparable results were nefazodone 55%, CBASP 52% and combined treatment 85%. (New England Journal of Medicine, May 18, 2000, “A comparison of nefazodone, the cognitive behavioral-analysis system of psychotherapy, and their combination for the treatment of chronic depression,” Keller, M B et al.)

Belviq® is an example of a de facto combination treatment in a single medication as noted in the package insert and accompanying advertising information for Belviq (Lorcaserin hydrochloride), “A Pharmacotherapeutic Option for Chronic Weight Management,” manufactured by Eisai Co., Ltd (BELV0122 Brochure, May 2013). Belviq is described as “indicated as an adjunct to a reduced-calorie diet and increased physical activity for chronic weight management in adults with an initial body mass index (BMI) of 30 kg/m2 or greater (obese) or 27 kg/m2 or greater” in the presence of certain risk factors.

All subjects in the study, whether on Belviq or placebo, had Lifestyle Management counseling. According to the information from Eisai, who now owns this compound, when they tested Belviq against placebo (using Arena Healthy Lifestyle Program [Data on File 44 at Eisai]), the following occurred:

All counselors were trained and certified to properly conduct the program. This program ensured consistency across all studies and study sites.

The objectives of the program were to:
Develop a moderate-intensity weight management program for all study participants
Standardize the weight management program across all study sites
Maximize patient recruitment and retention
Maintain counselor motivation

The initial counseling session visit took place on the day of randomization and included a Baseline Interview with the patient, assessment of the patient's dietary habits and activity level, and calculation of the patient's specific calorie requirement. The prescribed diet was approximately 600 calories less per day than the patient's calculated Estimated Energy Requirement (EER). The EER was calculated using WHO criteria with a fixed activity factor of 1.3 for most patients and an activity factor of 1.4 for patients who engage in >1 hour per day of aerobic exercise. At the conclusion of the initial visit, the patient was provided with materials including a Food & Activity Plan and Log and a pedometer.

Between visits, patients were asked to complete the Food & Activity Log, which they returned at each study visit for assessment by the counselor for compliance with the program. Non-compliance was addressed by the counselor, as appropriate, through additional discussion, education, and materials.

At each subsequent study visit, the Arena Healthy Lifestyle Program guided the counselor and patient through various preselected topics related to successful long-term weight management.

The above information shows the intensity of an adjunctive treatment used in the approval process of a medication but not necessarily subsequently used in any systematic way as part of the prescribing process of the medication. The adjunctive treatment used in the approval process of this medication is not even available to prescribing physicians. This is said with reasonable certainty as according to Eisai's own information: “As of May 2013, a computerized search of the medical literature using EMBASE, MEDLINE, BIOSIS, SCISEARCH, and IPAB did not identify any published information that discusses BELVIQ and the Arena Healthy Lifestyle Program®.

What can be learned from this approach to treatment combination, in this case medication with a regimen of diet and exercise? The literature from Eisai regarding Belviq suggests the following (Challenges for Healthcare Providers and Patients (BELV0122 Brochure, page 8)):

Combatting overweight or obesity and its associated medical, social, and economic consequences starts with the healthcare providers. It is imperative that all medical professionals be educated on overweight or obesity and appropriate courses of treatment, as well as how to accurately convey this information to patients. [Educate providers and convey information to patients—Belviq lesson 1]

The idea that overweight or obesity is a “disease” has only recently become prominent in the medical community. With the identification of risk factors and the definition of patient screening parameters comes the responsibility to discuss appropriate treatment options. [Providers need to discuss treatment options—Belviq lesson 2]

Patient motivation is a key component for success in a weight loss program. [Patient motivation is needed—Belviq lesson 3]

Discussing weight management with patients can be extremely challenging. Overweight or obese patients may avoid seeking medical care due to shame or concern about the associated stigma. [Patients avoid—Belviq lesson 4]

It also can be difficult for the physician to initiate a discussion of weight management for fear of triggering negative patient reactions. [Doctors may avoid—Belviq lesson 5]

Furthermore, physicians who are themselves overweight or obese tend to avoid the topic of weight management with their patients. It is the responsibility of healthcare providers and the medical community as a whole to create an environment that eases these fears and concerns on both sides and encourages communication. Patient motivation is a key component for success in a weight loss program, and a patient's primary healthcare provider can play a large role in helping to maintain this motivation. [Motivation from and for both health care providers and patients is required—Belviq lesson 6]

According to Eisai, once the issue of weight management has been brought to the attention of the patient, the next step is to set realistic goals and to outline a plan to achieve them. Some factors to consider when setting goals include:
1. Weight loss at the rate of 1 to 2 pounds per week (with a calorie deficit of 500 to 1000 kcal/day) commonly occurs for up to 6 months
2. The rate of weight loss usually declines after 6 months due to lesser energy expenditure at the lower weight
3. A patient's goals should be individually determined based on factors such as comorbidities and current lifestyle

It is important to set individualized, realistic goats for each patient because achieving predetermined goals is an important factor in maintaining patient motivation.

There are several weight management strategies available to doctors and patients. Each therapy is appropriate for a distinct patient set and has a different safety profile associated with it. Lifestyle modification is advised for overweight or obese patients with BMI greater than or equal to 25 kg/m2, though choosing a form of intervention can be difficult. Patient motivation is a key component for success in a weight loss program; it is a critical factor in choosing an appropriate therapy. Practitioners need to assess the patient's motivation to enter weight loss therapy, assess the readiness of the patient to implement the plan, and then take appropriate steps to motivate these patients.

Lifestyle modification, pharmacotherapy, and surgery are options that should be considered for certain patient sets based on BMI and past and current medical conditions. Social and historical factors influence attitudes toward the weight management therapies among both healthcare providers and patients. Many overweight or obese patients, particularly women, are more likely to try fad diets and over-the-counter weight loss supplements rather than a balanced diet and an exercise program. In addition, recent history has seen several pharmacologic weight loss agents enter the market only to be recalled due to safety concerns. This can lead to a hesitation to use other medical weight management treatment options, particularly pharmacotherapies. It is important to be aware of all available and appropriate therapies for each patient, and the associated clinically proven benefits as well as risk profiles.

The above information regarding Belviq from Eisai directly adds to or expands on the following:
1. Sometimes it is difficult to give patients the information that they need.
2. It is a doctor's responsibility to provide the information that patients need.
3. Doctors need to accurately convey information to patients.
4. Doctors avoid conveying information to avoid upsetting patients.
5. Doctors avoid conveying information to avoid upsetting themselves.
6. Patients avoid discussing certain topics to avoid upsetting themselves.
7. It is the responsibility of healthcare providers and the medical community as a whole to create an environment that eases these fears and concerns on both sides and encourages communication.
8. Patient motivation is a key component for success in a weight loss program
9. A patient's primary healthcare provider can play a large role in helping to maintain/grow a patient's motivation.
10. Once the issue of weight management has been brought to the attention of the patient, the next step is to set realistic goals and to outline a plan to achieve them.
11. The FDA approved Belviq as a pharmacotherapeutic option as part of a combined treatment as described in the brochure: Lifestyle modification is advised for overweight patients with a BMI 24 to 26.9 kg/m²; Lifestyle modification with pharmacotherapy is advised for overweight patients with a BMI 27 to 29.9 kg/m²; lifestyle modification with pharmacotherapy and surgery is advised for obese patients with a BMI of 30 kg/m² or greater, though choosing a form of intervention can be difficult.
12. Patient motivation is a key component for success in a weight loss program and it is a critical factor in choosing an appropriate therapy.
13. Practitioners need to assess the patient's motivation to enter weight loss therapy, assess the readiness of the patient to implement the plan, and then take appropriate steps to motivate these patients.
14. It is important for practitioners to be aware of all available and appropriate therapies for each patient.

The FDA-approved BELV0122 Brochure indicates that lifestyle modification is advised for overweight or obese patients with BMI greater than or equal to 25 kg/m2, though choosing a form of intervention can be difficult. In particular, what is not addressed is how to make the lifestyle modification intervention simpler, easier, more comfortable, more often, with more authority, with more knowledge and/or more appropriately.

As noted above, at this time, the present state of the art of medicine is such that it is difficult for a health care practitioner, even after he or she agrees with the idea of augmenting the treatment plan with a lifestyle modification, to know what lifestyle modification to choose. Some of the reasons that lifestyle modifications are not recommended by physicians and thus the care for patients is less than desirable, less than acceptable, more invasive than is needed and/or less than optimal include but are not limited to the following deficiencies in the practice/thinking of physicians that interfere with the physician prescribing a lifestyle modification:
1. What are lifestyle modifications?
2. How can I teach these to my patients?
3. What brochures are available?
4. How much do lifestyle modifications cost me or my patients?
5. If I cannot teach the lifestyle modification to my patients, who can?
6. Who should teach the lifestyle modification to my patients?
7. What is to be expected from the lifestyle modification?
8. When should the lifestyle modification be practiced?
9. How can the patient remember to implement the lifestyle modification?
10. What lifestyle modifications are appropriate for this patient?
11. What lifestyle modifications are appropriate for any patient?
12. What am I really recommending for this patient?
13. What if the patient doesn't like the lifestyle modification recommendation?
14. Can there be adjustments made to the lifestyle modification?
15. Is there any evidence that the lifestyle modification is beneficial?
16. If I cannot teach the lifestyle modification how will the patient learn it?
17. How can I make adjustments to the lifestyle modification?
18. How can I make adjustments to the lifestyle modification based on patient input?
19. How can I make adjustments to the lifestyle modification based upon the information available to me?
20. How can I make adjustments to the lifestyle modification based on patient input and the information available to me?

Whether treating mental illness, problems with drug abuse, alcohol abuse, pain, appetite, cigarette smoking, tobacco use, anxiety, depression, quality-of-life, cancer, asthma, urinary dysfunction, behavioral disturbance of senile dementia of the Alzheimer's type or a variety of physical and mental ailments, etc., there continues to be a dichotomy between what are referred to as complimentary/alternative (adjunctive) medical treatments and the primary standard (allopathic) medical treatments. This dichotomy is, unfortunately, the state-of-the-art.

The National Institutes of Health (NIH) reported that in 2007 Americans spent $4 billion on mindfulness-related alternative treatments alone. Despite the fact that there are thousands of apps (computer-based applications) available for mindfulness and other alternative medical treatments, what continues to be missing is the ability to integrate the allopathic treatment/somatic treatments and the alternative/complementary treatments together in a way that promotes efficacy, compliance and adherence.

As noted above, some pharmaceutical entities require integrating the two types of treatment (the allopathic treatment and the alternative/complementary treatment) by including lifestyle management for all patients in their clinical research studies of allopathic treatments. This includes antihypertensive, lipid lowering, glucose lowering or weight loss proof of concept or phase I, II, III or IV pharmacological regimen studies and others.

The premises set forth immediately hereabove, including the difficulty of implementing an adjunctive regimen, are supported in the article “Management of Obesity in Primary Care” by Laurey R. Simkin-Silverman, Rena R. Wine, OBESITY RESEARCH, Vol. 5 No. 6 Nov. 1997, 603, which is quoted herebelow (with footnotes omitted).

“Although physicians acknowledge that obesity is important to treat, clinicians do not adequately counsel their patients who are overweight because of barriers to health behavior counseling. Commonly cited barriers among physicians in general include lack of time, lack of training, limited staff support, and limited access to patient-educational materials. Moreover, there are unique obstacles to obesity counseling in that physicians may have negative attitudes toward patients with obesity and may blame patients for their inability to maintain weight loss. At least one recent study has attempted to modify these negative attitudes by implementing an educational program for medical students.

Although physician-delivered obesity counseling has received little attention, training physicians to counsel their patients to reduce other cardiovascular risk behaviors such as smoking, sedentary lifestyle, and high-fat diet have been studied. Although Continuing Medical Education approaches have been largely unsuccessful in changing physician practice or patient behavior, a growing number of studies suggest that office-based interventions including behavioral training for physicians and staff, clinical tools to prompt and facilitate counseling, and the provision of theoretically based patient materials can be effective. Moreover, patient-centered counseling approaches have been developed to assist physicians. One model based on the National Cancer Institute's smoking cessation manual for physicians has been adapted for use in physical activity counseling. Physicians are prompted to counsel using the following five steps: (1) ask (or address the agenda), (2) assess, (3) advise, (4) assist, and (5) arrange follow-up. The office-based training approach and the use of this five-step model were modified for use in this study.

Physicians received two hours of individual training conducted at the medical office setting. During training, they were given a treatment manual and patient self-help materials. Physicians were encouraged to assess BMI and body fat distribution (in addition to weight), emphasize to patients the medical benefits of modest weight loss, set specific and realistic weight and exercise goals, emphasize caloric restriction and dietary fat reduction, give exercise recommendations specific to weight control, and provide patients with a weight control prescription and behavioral handouts. One important aspect of giving patients advice to lose weight is related to setting specific and appropriate weight loss goals. During training, physicians were encouraged to start patients off with a weight loss goal of 5 lbs to 25 lbs, as opposed to referring to ideal weight charts because modest weight loss is associated with improved risk factors and is more realistic for patients to achieve.”

Other attempts have been made to simulate multimodal treatment, but as referenced above, the aspects of a multimodal treatment that are non-pharmacologic are difficult for physicians to choose. Nevertheless, at the present state of pharmacologic development, patients are not enrolled in a clinical research study, for example, in a smoking cessation, without being offered support staff that provides educational information and/or psychosocial support. This study design of pharmacologic development and the one-on-one instruction offered at a clinical research site is different from the much less personal and much less effective state-of-the-art treatment in actual practice wherein FDA approved medications are dispensed as pharmacological agents only despite being approved after being studied within multi-modal treatment. That is, after FDA approval no more than lip service is paid to the psychosocial interventions of diet and exercise, etc. The presumed psychosocial support that was taken into the equation when the medication was FDA approved is either available only on the internet, inside a booklet, via a toll free number, via a third party or not at all.

This ongoing dichotomy between the two approaches, allopathic (pharmacological treatments) and alternative/complementary (adjunctive) treatment, results in less than optimal outcomes for patients. For instance, the aforementioned Multi-Center Treatment Approaches to ADHD study (MTA) was first marketed by pharmaceutical companies as proving that medication alone was the most important factor in treatment. The pharmaceutical companies lectured doctors about how much better three times a day medication (or medication throughout the day) was than either the standard community treatments or psychosocial support. Only over time did the various pharmaceutical companies verbalize the correct conclusion, namely, that the optimal treatment was proven to be a combination of medication plus psychosocial support.

The dichotomy continues, however. A Journal of the American Medical Association (JAMA) article dated Feb. 19, 2014, contained an article entitled “Effect of Citalopram on Agitation in Alzheimer Disease.” The study design in the article was as follows: “participants (N equals 186) were (all) randomized to receive the same psychosocial intervention. Then they also received either Citalopram (N equals 94) or placebo (N equals 92) for 9 weeks.”

This was a study whose primary objective was to evaluate the efficacy of citalopram for agitated patients with Alzheimer's disease, yet every patient received a psychosocial intervention, something that might not be available to 186 actual outpatients with Alzheimer's disease (and their families). The fact that the benefit of psychosocial interventions alone was a factor in the final outcome was noted by some of the critical reviewers of the article. In other words, the benefit found was not from Citalopram alone.

It almost always takes less time for a medical doctor, osteopathic physician, physician's assistant, nurse clinician, or any prescribing provider to prescribe a pill than to provide psychosocial support. This means that psychosocial support is not available at all or only available through a third party, such as another health provider, another non-healthcare professional or available via booklet, pamphlet, the internet, or a smart phone application. When there is separate/divided treatment, i.e., when one provider is not closely managing, prescribing, providing, evaluating or involved with all of the treatments offered, suggested or needed, the follow-through (adherence/compliance) to any less closely supervised portion of the regimen is even more difficult to ensure. Equal and/or consistent follow-through when there are two regimens to follow (multimodal treatment options), even when the regimens are synergistic, might be less likely, given any type of separation in time or space between where the two or more different regimens originate and where the two different regimens are delivered.

While there is available diabetes medication, pain medication, mental health medication, blood pressure medication, antismoking medication, asthma medication, and medication for problems with drug abuse, alcohol abuse, pain, appetite, cigarette smoking or tobacco use, anxiety, depression, quality-of-life, cancer, asthma, urinary dysfunction, behavioral disturbance of senile dementia of the Alzheimer's type and a variety of physical and mental ailments, etc., alternative approaches to supplement medication over the years have included the following or could include the following: the power of suggestion; the power of the health care provider; abdominal/diaphragmatic breathing; the relaxation response; hypnosis; mindfulness; exercise; diet; heart rate variability training; autogenic training (AT) using all six modes or AT using just heaviness, warmth, heart beating calmly and abdominal warmth training; progressive muscle relaxation; mindfulness based stress reduction (Kabat-Zinn, 1990); biofeedback; neurolinguistic programming; visualization; note taking; a self-help version of applied relaxation; mindfulness focused on what a patient is presently noticing, thinking, feeling, and doing; stretching; abbreviated progressive relaxation training (APRT); yoga; imagery; positive self-talk; packet journaling; breathing exercise; meditation/mindfulness; reduced arousal training; R-state training; other alternative/complimentary treatments that can be delivered as light, sound, energy, visual cues, auditory, or in ways not yet discovered that are closer to these methods than a pharmaceutical intervention, or a combination of all or some of the above.

Despite the large number of alternative approaches, little research has been done on combining one of the relaxation techniques or alternative/complementary (adjunctive) techniques with another, let alone integrating any of these relaxation techniques or alternative/complementary techniques with medication.

There is evidence that many of these relaxation techniques or alternative/complementary treatments are individually successful without the addition of any pharmacological agents. According to Lehrer, et al., In Principles and Practice of Stress management, 3rd Edition, 2007, The Guilford Press, New York, N.Y., treatments using heart rate variability training (HRV) have proven to be efficacious for asthma and hypertension. Stress and tension reduction is helpful with anxiety states, panic disorder, phobic disorders, neurotic depression, and other disorders such as irritable bowel syndrome with vomiting, diarrhea and constipation, bruxism, essential hypertension, coronary heart disease, rheumatological pathologies, chronic fatigue and such pains as those of headaches and backaches. Evidence has been shown regarding the benefit of progressive muscle relaxation in the treatment of “psychiatric tension pathologies,” as well as nervous hypertension, insomnia, generalized anxiety disorder, cardiac neurosis, chronic insomnia, cyclothymic disorders, obsessive-compulsive disorder, hypochondria, fatigue states, dysthymia, esophageal spasm, convulsive tics, various bowel disorders, arterial hypertension, tension headaches, pain, headache, generalized stress, and asthma.

APRT (Abbreviated Progressive Muscle Relaxation) clinical indications include anxiety and stress, hypertension, tension headaches, asthma, chronic pain, quality-of-life and cancer patients, PTSD, improved sleep quality, urinary dysfunction and behavioral performance in Alzheimer's.

Although the FDA in the past has required some treatments to be studied concomitantly before they are approved, e.g., Prozac and Zyprexa were tested together for efficacy and safety before receiving approval to be marketed as Symbyax, the FDA has both covertly and tacitly approved some treatments without further study. Such is the case for the lifestyle management approved with Belviq, psychosocial interventions approved with agents for smoking cessation and for medical foods approved for distribution by prescription without further clinical trials. Since the FDA did not require approval of the dietary and exercise regimens required for subject participation in the Belviq studies noted above, it is suspected that like medical foods that have been so well studied that no further testing is needed, some or all adjunctive measures, e.g., verbal instructions, progressive relaxation technique, the relaxation response, diaphragmatic breathing, and others, will be permied to be delivered as adjuncts to approved pharmaceuticals without further study.

### SUMMARY OF THE INVENTION

The present invention provides a system and method for multimodal medical treatment optimization. The system and method involve (1) providing a patient with a primary medical treatment via a first delivery system, (2) providing a patient with an adjunctive medical treatment via a second delivery system built into, incorporated within or integrated with the first delivery system, and (3) adjusting at least one of the primary medical treatment and the adjunctive medical treatment as a result of the patient's response to at least one of the primary medical treatment and the adjunctive medical treatment. The subject application provides several non-limiting examples of primary and adjunctive medical treatments, first and second delivery systems, and means by which the second delivery system may be incorporated into or integrated with the first delivery system.

The instant invention focuses on a new system and method of combining different evidence-based or anecdotally-useful procedures and/or treatments. The invention produces the following benefits:
1. money savings in part due to improved adherence
2. money savings in part due to improved functioning
3. improved functioning independent of improved adherence

These benefits are achieved as follows:

1. Improved adherence to the medication regimen
2. Improved adherence to the complementary and alternative (adjunctive) approaches, stress management techniques, psychotherapeutic techniques and other procedures, techniques, rating scales, diagnostic tools, monitoring tools, monitoring devices, etc.
3. Improved adherence to both the medication regimen and the complementary and alternative (adjunctive) approaches, stress management techniques, psychotherapeutic techniques and other procedures, techniques, rating scales, diagnostic tools, monitoring tools, monitoring devices, etc.
4. Intelligent adjustments of the medication regimen (primary treatment) based upon information about adherence and functioning provided by the patient, information about the patient's condition, health care practitioner(s) observations of the patient's condition, other information available to the health care practitioner(s), etc.
5. Intelligent adjustments of adjunctive treatments including complementary and alternative (adjunctive) approaches, stress management techniques, psychotherapeutic techniques and other procedures, techniques, rating scales, diagnostic tools, monitoring tools, monitoring devices, etc., based upon adherence and functioning provided by the patient, information about the patient's condition, health care practitioner(s) observations of the patient's condition, information available to the health care practitioner(s), etc.
6. Coupling the adjunctive treatments with the primary medical treatment such that they are contiguous in time and space with the dispensing, ingesting, delivery or use of the primary medical treatment for the promotion of adherence/compliance, efficacy and/or other factors of the primary medical treatment and/or the adjunctive medical treatment(s).

The subject invention integrates two or more different regimens, the standard pharmaceutical (primary medical treatment) regimens and the complimentary/alternative (adjunctive) treatments in a way that has not been proposed, used or developed before.

According to first aspect, the subject application includes a system for multimodal medical treatment comprising a first delivery system for delivering a primary medical treatment to a patient and a second delivery system integrated with the first delivery system. The second delivery system includes a memory having an audio file, a video file or an audiovisual file of an adjunctive medical treatment, and an output delivery device operatively connected to the memory for delivering the adjunctive medical treatment to the patient.

The first delivery system according to the first aspect comprises a medication containing device.

The medication containing device according to the first aspect may comprise a patch.

The medication containing device according to the first aspect may comprise an inhaler.

The output delivery device according to the first aspect is a screen, earphones, a speaker, or a holographic projection.

The output delivery device according to the first aspect executes the audio file, the video file or the audiovisual file.

According to a second aspect, the subject application provides a method for multimodal medical treatment optimization comprising the steps of providing a patient with the multimodal medical treatment optimization system of the first aspect, delivering the primary medical treatment via the first delivery system, delivering the adjunctive medical treatment via the second delivery system; and adjusting at least one of the primary medical treatment and the adjunctive medical treatment as a result of the patient's physiological or psychological response to at least one of the primary medical treatment and the adjunctive medical treatment.

According to a third aspect, the subject application provides a system for multimodal medical treatment comprising a first delivery system for delivering a primary medical treatment to a patient, and a second delivery system integrated with the first delivery system, the second delivery system including an adjunctive medical treatment comprising at least one of light therapy, aromatherapy and magnetic therapy.

The first delivery system according to the third aspect comprises a medication containing device.

The second delivery system according to the third aspect may comprise an aerosol dispensing device attached to the first delivery system.

The second delivery system according to the third aspect may comprise a light illuminating device attached to the first delivery system.

The second delivery system according to the third aspect may comprise a magnet attached to the first delivery system.

According to a fourth aspect, the subject application provides a multimodal medical treatment system comprising a medication containing device for housing a primary medical treatment, and an audiovisual module having stored audio, video or audiovisual files of an adjunctive medical treatment, wherein when the medication containing device is activated, playback of the audio, video or audiovisual files begins.

The audiovisual module according to the fourth aspect includes an output delivery device.

The output delivery device according to the fourth aspect is a screen, earphones, a speaker, a USB port or a holographic projection.

The screen according to the fourth aspect is an LED or LCD screen.

The screen according to the fourth aspect is extendable and retractable.

According to a fifth aspect, the subject application provides a multimodal medical treatment system comprising a medication containing device for housing a primary medical treatment, wherein the medication containing device includes an integral breathing rate controlling tool.

The tool according to the fifth aspect may comprise a timepiece

The tool according to the fifth aspect may comprise an adjustable dial.

Other details, objects and advantages of the present invention will become apparent as the following description of the presently preferred embodiments and presently preferred methods of practicing the invention proceeds.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a plan view of a daily pill dispenser coupled with a device for enabling a user to receive an audio message concerning an adjunctive treatment;

FIG. 2 is a perspective view of a medication bottle coupled with a cable for connecting the bottle to computer or similar device whereby the computer or similar device may provide the patient with an adjunctive treatment or instructions therefor or vice versa;

FIG. 3 is a perspective view of a medication bottle coupled with a digital voice recorder for enabling a user to receive an audio message concerning an adjunctive treatment or for an audio message or other file to be downloaded onto the medication bottle for use when medication is delivered to the patient;

FIG. 4 is a perspective view of a medication bottle coupled with a sound producing device for enabling a user to receive an audio message concerning an adjunctive treatment;

FIGS. 5A-5C are sequential views illustrating the release of a listening device which is incorporated into the cap of a medication bottle;

FIG. 6 is a front elevation view of a medication bottle having a cap providing for the adjustment of controlled breathing as an adjunctive treatment;

FIG. 7 is a perspective view of a medication bottle equipped with audio file generating circuitry and a device for enabling a user to receive an audio message concerning an adjunctive treatment from the audio file generating circuitry;

FIG. 8 is a perspective view of a medication bottle equipped with audio file generating circuitry and a device for enabling a user to receive an audio message concerning an adjunctive treatment pertaining to stress relief from the audio file generating circuitry;

FIG. 9 is a perspective view of a medication bottle equipped with audio file generating circuitry and a device for enabling a user to receive an audio message concerning an adjunctive treatment pertaining to confidence building from the audio file generating circuitry;

FIG. 10 is a perspective view of a medication bottle equipped with audio file generating circuitry and a device for enabling a user to receive an audio message concerning an adjunctive treatment pertaining to relaxation techniques of differing duration from the audio file generating circuitry;

FIG. 11 is perspective view of a medication bottle having a cap equipped with a LED screen or LCD screen such as a timepiece or otherwise for assisting in controlled breathing as an adjunctive treatment;

FIG. 12 is a plan view of a starter titration package for new medication coupled with a device for enabling a user to receive an audio message concerning an adjunctive treatment;

FIG. 13 is a perspective view of an inhaler device coupled with a listening device for enabling a user to receive an audio message concerning an adjunctive treatment;

FIG. 14 is a perspective view of a medication patch coupled with a listening device for enabling a user to receive an audio message concerning an adjunctive treatment;

FIG. 15 is a perspective view of a box containing medication patches coupled with at least one listening device for enabling a user to receive an audio message concerning an adjunctive treatment;

FIG. 16 is a front elevation view of a medication bottle atop which is provided an aerosol dispenser for delivering an adjunctive treatment in aerosol form;

FIG. 17 is a front elevation view of a medication bottle including a mechanism for generating a holographic image for delivering an adjunctive treatment message;

FIG. 18 is a perspective view of a medication bottle including a light source for delivering an adjunctive treatment involving light;

FIG. 19 is a front elevation view of a medication bottle coupled with magnets for delivering an adjunctive treatment involving magnetism;

FIG. 20 is a front elevation view of a medication bottle equipped with an electrical port for enabling connection to a computer or similar device for delivering an adjunctive treatment;

FIG. 21A is a front elevation view of a medication bottle equipped with a flexible electronic screen in an extended state for delivering a visual adjunctive treatment message;

FIG. 21B is a view of the medication bottle of FIG. 21A with the flexible electronic screen in a retracted state;

FIG. 22 is a perspective view of a squeezable medication tube coupled with at least one listening device for enabling a user to receive an audio message concerning an adjunctive treatment;

FIG. 23A is a front elevation view of a medication bottle equipped with a Bluetooth enabled ear engaging device for enabling wireless connection to a computer or similar device for delivering an adjunctive treatment;

FIG. 23B is a view of the interior of the cap of the medication bottle of FIG. 23A; and

FIG. 23C is a view of the cap of the medication bottle of FIG. 23A shown as it would be worn by an ear of a patient.

### DETAILED DESCRIPTION OF THE INVENTION

Reference will now be made in detail to the present embodiments of the invention illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, above, below and diagonal, are used with respect to the accompanying drawings. The term “distal” shall mean towards the bit-end. The term “proximal” shall mean towards the backhead-end. Such directional terms used in conjunction with the following description of the drawings should not be construed to limit the scope of the invention in any manner not explicitly set forth.

Certain terminology is used in the following description for convenience only and is not limiting. The words “right,” “left,” “lower” and “upper” designate directions in the drawings to which reference is made. The words “inwardly” and “outwardly” refer to directions toward and away from, respectively, the geometric center of the identified element and designated parts thereof. Additionally, the term “a,” as used in the specification, means “at least one.” The terminology includes the words noted above, derivatives thereof and words of similar import.

The following definitions and discussion of general principles are offered to provide context for the subsequent detailed description of representative embodiments of the invention.

As used herein, “medical treatment” or words of similar effect, whether used in connection with a primary medical treatment or adjunctive medical treatment(s), shall mean proven or unproven (unapproved, novel, unique or other) medical techniques, medication, medical foods, orphan drugs, procedures, diagnostic tools, diagnostic procedures, surgical procedures, over the counter treatments, nutraceuticals, treatments under investigation, alternative medicine treatments, complementary medicine treatments or approaches, stress management techniques, psychotherapeutic techniques and other physiological or psychological procedures, techniques, rating scales, tools or treatments to promote adherence, efficacy or other factors related to either or both of the primary medical treatment and the adjunctive medical treatment(s).

According to the present invention, first medical treatment delivery systems for delivering a primary medical treatment may include but are not limited to:
1. a medication vial
2. a dispensing card for a medication including unit dose medication
3. a blister pack holding medication
4. a rectangular dispensing card for a medication including unit dose medication and combination medications
5. a circular dispensing card/wheel for a medication including unit dose medication and combination medications
6. a medication container holding pills or capsules
7. a dispensing container
8. any device in which medication is placed or dispensed
9. any device in which over the counter (OTC) vitamins, nutraceuticals, minerals, health promoting elixirs or the like is placed or dispensed
10. any device in which a medical food is placed or dispensed
11. any device in which an orphan drug is placed or dispensed
12. any device in or from which a medication is placed that will be dispensed nasally
13. any device in or from which an aerosol medication is placed that will be dispensed via insufflation
14. any device in or from which an investigational medication is placed or dispensed
15. any container of any size or shape holding a gas, liquid or solid or a mixture of gases, liquids and/or solids that are a drug, a food, a food ingredient, a dietary supplement or a health related ingredient
16. any container of any size or shape holding a gas, liquid or solid or a mixture of gases, liquids and/or solids from which a drug, a food, a food ingredient, a dietary supplement or a health related ingredient is dispensed
17. any combination of gas, liquids or solids to deliver a medication intravenously, intramuscularly, subcutaneously, vaginally, rectally, directly into the stomach, directly into the intestines, directly into the bladder, directly into the lungs, directly into a body part, directly into a nebulizer or directly into the air
18. any combination of gas, liquids or solids to remove solids, liquids or gases from the body
19. any intervention or treatment that removes energy from the body, delivers energy to the body or measures energy in the body
20. any intervention or treatment that removes, delivers or measures body parts
21. any intervention or treatment that removes, delivers, or measures the content of the body
22. any intervention or treatment that removes, delivers, or measures what is being excreted from or delivered to the body
23. any combination of gases, liquids or solids that removes, delivers, or measures a medication sublingually, orally, transdermally or via a catheter
24. any book or electronic book that delivers a medication or treatment after a patient reads it or while it reads to a patient
25. any electronic device that delivers a medication or treatment to a patient
26. any device that delivers a medication or treatment that counts medication
27. any device that dispenses, contains, removes, delivers, or measures medication
28. any device that delivers a medication or treatment via a catheter
29. any other methods that remove, deliver, or measure a medical intervention or treatment including x-ray, CAT Scan, MRI, PET scanners, laser, ultrasound equipment, pens, pencils, gowns, Intravenous lines, feeding lines, PEG insertion, TMS, Stress test, blood work, catheter lines, pole to hold intravenous lines, intra-arterial lines, drainage lines, drainage material, bandages, dressings, sterile patches, non-sterile patches, radioactive materials, nuclear medicine equipment, chiropractic equipment and treatments, podiatric equipment and treatments, occupational therapy equipment and treatments, nursing equipment and treatments, physical therapy equipment and treatments, physician assistant equipment and treatments, medical equipment and treatments, surgical equipment and treatments, non-medicated patches, medicated patches, medically related tubing, medically related packing material, medically related furniture, medically related machinery, medically related transportation equipment, wheel chairs, medically related stents, biofeedback equipment, biofeedback machinery, any device that aids in diagnosis, any device that aids in treatment, any device that aids in health promotion, needles, scalpels, sutures, syringes, trochanters, scissors, gowns and beds
30. any pill dispensers that are designed to make sure a patient takes or tracks that a patient takes his/her medication
31. any devices that are designed to make sure a patient takes or tracks that a patient takes his/her medication
32. any devices that are designed to improve adherence
33. any paper that contains or delivers medication
34. any stent that contains or delivers medication
35. any device for splinting teeth, bone, etc.
36. any device for splinting teeth, bone, etc., that contains or delivers medication
37. any cast or material to put on a cast that contains or delivers medication
38. any devices that are designed to improve adherence
39. any devices that are designed to improve efficacy
40. any devices that are designed to measure the content of the body

According to the present invention, adjunctive treatments may include the following proven or unproven complimentary/alternative treatments, etc., including but not limited to the following physiological, psychological and other treatments:

1. abdominal/diaphragmatic breathing
2. the relaxation response
3. hypnosis
4. mindfulness
5. exercise
6. diet, diet advice and/or meal planning
7. heart rate variability training
8. autogenic training (AT) using all six modes
9. autogenic training (AT) using just heaviness, warmth, heart beating calmly and abdominal warmth training
10. progressive muscle relaxation
11. mindfulness based stress reduction
12. biofeedback
13. neurolinguistic programming
14. visualization
15. note taking
16. self-help version of applied relaxation
17. mindfulness focused on what a patient is presently noticing, thinking, feeling, and doing
18. stretching
19. abbreviated progressive relaxation training (APRT)
20. yoga
21. imagery
22. positive self-talk
23. packet journaling
24. breathing exercise
25. meditation
26. reduced arousal training
27. R-state training
28. motivational interviewing
29. nutritional counseling
30. stress management
31. autogenic biofeedback training
32. neurofeedback
33. exercise therapy
34. whole body breathing
35. Qigong therapy or medical qigong
36. eye movement desensitization and reprocessing (EMDR)
37. cognitive approaches
38. stress inoculation training
39. bibliotherapy
40. writing therapy
41. behavioral activation therapy
42. psychotherapy
43. cognitive behavioral therapy
44. social skills training
45. psychoeducation
46. family psychoeducational therapy
47. interpersonal therapy
48. exposure and response prevention therapy
49. behavior therapy
50. self-administered exposure therapy
51. magnets and magnet therapy
52. supportive psychotherapy
53. cheerleading therapy
54. music therapy
55. sound therapy
56. light therapy
57. aroma therapy
58. medication
59. homeopathic regimens
60. alternative medicine treatments
61. complementary medicine treatments
62. cognitive behavioral modeling
63. cognitive restructuring
64. transcranial therapy
65. directions for correctly implementing proven or unproven medical techniques, medication, medical foods, orphan drugs, procedures, diagnostic tools, diagnostic procedures, surgical procedures, over the counter treatments, nutraceuticals, treatments under investigation, adjunctive treatments, alternative medicine treatments, complementary medicine treatment, tools or techniques to promote adherence
66. all the above in a variety of languages
67. all the above in a variety of voices including, without limitation, the voice of the patient's health care practitioner or even strangers who may compliment the patient for undergoing the adjunctive treatment
68. all the above in a variety of voices including a family member's voice
69. all the above in a variety of voices including the patient's own voice
70. new treatments
71. other treatments
72. electronic personal health record
73. biofeedback including EEG, heart rate monitoring, blood pressure monitoring, galvanic skin response, temperature, strain gauges or digital feedback
74. transcendental meditation
75. family therapeutic, supportive and integration techniques
76. receive information in the form of news, entertainment, literature, sports, etc., that may be in common with others not taking the primary medical treatment or a different primary medical treatment

According to the present invention, second medical treatment delivery systems for delivering adjunctive medical treatment(s) may include but are not limited to:
1. in the cap of a medication vial
2. in the cap of a medication vial containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
3. in the bottom of a medication vial
4. in the bottom of a medication vial containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
5. in the body of a medication vial
6. in the body of a medication vial containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
7. in the dispensing card for a medication including unit dose medication and combination medications
8. in the dispensing card for a medication including unit dose medication and combination medications containing an MP3, a sound card, a chip, (e.g., an MP3 digital recording, WMA digital recording, other digital recording, sound card, computer memory or computer chip) or some other way to record and/or deliver an analog, digital or media audio, video, audiovisual or other type of file
9. in the vial
10. in the vial holding pills or capsules
11. in the vial holding pills or capsules containing an MP3, a sound card, a chip, (e.g., an MP3 digital recording, WMA digital recording, other digital recording, sound card, computer memory or computer chip) or some other way to record and/or deliver an analog, digital or media audio, video, audiovisual or other type of file
12. in the dispensing container
13. in the dispensing container containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
14. in any device in which medication is placed or dispensed containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
15. any device in which over the counter vitamins, nutraceuticals, minerals, health promoting elixirs or the like is placed or dispensed containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
16. any device in which a medical food is placed or dispensed containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
17. any device in which an orphan drug is placed or dispensed containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
18. any device in or from which a medication is placed that will be dispensed nasally containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
19. any device in or from which an aerosol medication is placed that will be dispensed via insufflation containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
20. any device in which an investigational medication is placed or dispensed containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
21. the cap of any container of any size or shape holding a gas, liquid or solid form or a mixture of gases, liquids and/or solids that are a drug, a food, a food ingredient, or a dietary supplement containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
22. any container of any size or shape holding a gas, liquid or solid form or a mixture of gases, liquids and/or solids that are a drug, a food, a food ingredient, or a dietary supplement containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
23. any combination of gases, liquids or solids to deliver a medication intravenously, intramuscularly, subcutaneously, vaginally, rectally, directly into the stomach, directly into the intestines, directly into the bladder, directly into the air or directly into the lungs containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
24. any combination of gases, liquids or solids to remove gases, liquids or solids from the body containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
25. any treatment to remove energy from the body, to deliver energy to the body or to measure energy in the body containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
26. any treatment to measure body parts containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
27. any combination of gases, liquids or solids to deliver a medication sublingually, orally, transdermally containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
28. any combination of gases, liquids or solids to deliver a medication via a catheter containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
29. a book that delivers a medication after a patient reads it containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
30. an electronic book that reads to a patient as it dispenses medication
31. a catheter containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
32. x-ray, CAT Scan, MRI, IV, IM, intravenous lines, feeding lines, PEG insertion, TMS, Stress test, blood work, catheter lines
33. an audio/video projection device
34. holographic projection
35. a flexible LED or LCD screen that is part of a dispensing container
36. any of the above delivering an MP3 digital recording, WMA digital recording, other digital recording, or some other way to record and/or deliver an analog, digital or media audio, video, audiovisual or other type of file or delivering a second, third, etc. adjunctive treatment or method
37. any of the above delivering an MP3 digital recording, WMA digital recording, other digital recording, sound card, computer memory or computer chip delivering smell, light(s), color(s), containing a screen on which information can be viewed or written, have paper on which information can be viewed or written, have movement capabilities, delivering a massage, having a taste, having the ability to be a variety of temperatures, having the ability to apply pressure to a body part or an object, having the ability to deliver acupressure, having the ability to apply an electric shock, having the ability to elicit an aura, having the ability to change an aura, releasing energy in the form of light, sound, music or energy, taking a scale, meds adjusted according to score on a scale, taking a physiological test, meds adjusted according to score on a physiological test, taking a psychological test, meds adjusted according to score on a psychological test
38. via a telephone type of device integrated into the dispensing container that, for instance, could automatically dial a significant other upon taking medication to get support from others
39. any rating scale, monitoring tool, monitoring device, etc or the container in which the rating scale, monitoring tool, monitoring device, etc. is kept containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file
40. any complementary and alternative (adjunctive) approaches, stress management techniques, psychotherapeutic techniques and other procedures, techniques, etc. or the container in which any complementary and alternative (adjunctive) approaches, stress management techniques, psychotherapeutic techniques and other procedures, techniques, etc is kept containing an MP3, a sound card, a chip (e.g., an MP3 digital recording, WMA digital recording, other digital recording, sound card, computer memory or computer chip) or some other way to record and/or deliver an analog, digital or media audio, video, audiovisual or other type of file

According to the present invention, the adjunctive treatments, when and where they are appropriate to be delivered, can be delivered via the above devices in many ways, including but not limited to:
1. preprogrammed
2. programmable
3. activated upon opening
4. placed into a removal slot
5. placed into multiple removal slots
6. have multiple preprogrammed options
7. have the ability to be programmed by others or remotely
8. have the ability to receive the same adjunctive treatment, alternative medicine treatment, or complementary medicine treatment as other individuals.
9. have speakers to deliver the contents audibly
10. containing earphone
11. containing biofeedback devices
12. containing an LED or similar screen
13. activated upon use
14. containing patient preferences
15. containing doctor preferences
16. adjusted according to use
17. adjusted according to doctor preferences
18. adjusted according to patient preferences
19. adjusted according to doctor preferences based on response to medication
20. adjusted according to patient preferences based on response to medication
21. adjusted according to doctor preferences based on response to the complementary and alternative treatment
22. adjusted according to patient preferences based on response to the complementary and alternative treatment
23. adjusted according to doctor and patient preferences based on response to the complementary and alternative treatment
24. adjusted according to doctor and patient preferences based on response to the medication
25. adjusted according to doctor and patient preferences based on response to the medication and to the complementary and alternative treatment

According to the present invention, feedback from the delivery of the primary and adjunctive treatment(s) or modalities can be used to modify the delivery of, use of and/or integration of the treatments for multimodal treatment optimization as follows:
1. adjusted according to use
2. adjusted in regards to delivery method
3. adjusted according to doctor preferences
4. adjusted according to patient preferences
5. adjusted according to doctor preferences based on response to medication
6. adjusted according to patient preferences based on response to medication
7. adjusted according to doctor preferences based on response to the complementary and alternative treatment
8. adjusted according to patient preferences based on response to the complementary and alternative treatment
9. adjusted according to doctor and patient preferences based on response to the complementary and alternative treatment
10. adjusted according to doctor and patient preferences based on response to the medication
11. adjusted according to doctor and patient preferences based on response to the medication and to the complementary and alternative treatment
12. adjusted according to doctor and patient preferences and added to other prescriptions for that individual
13. adjusted according to doctor and patient preferences and those preferences can be integrated with those from other prescriptions for that individual

The present invention involves shared treatment and decision making via providing information to patients, obtaining patient preferences and aligning these to tailor communication to the patient. The adjunctive medical treatment(s) are delivered via second delivery systems that are incorporated/built into/juxtaposed the first delivery systems for the primary medical treatment in such a way as to be contiguous in time and space with the dispensing, ingesting, delivery or use of the primary medical treatment for the promotion of adherence/compliance, efficacy and/or other factors of the primary medical treatment and/or the adjunctive medical treatment(s).

As a result of use of integrated delivery systems described herein, there is the ability to gather information obtained through the use of the integrated system not limited to compliance/adherence and/or efficacy and to make changes in the proven, unproven and/or adjunctive treatments over time based on the following.

A medical intervention or treatment is delivered and a number of different complementary and/or alternative (adjunctive) treatment approaches, stress management techniques, psychotherapeutic techniques and other procedures, techniques, rating scales or diagnostic tools, etc. are delivered. The physical and/or emotional response to the different complementary and/or alternative treatment approaches, stress management techniques, psychotherapeutic techniques and other procedures, techniques, rating scales or diagnostic tools, etc. are reviewed by the patient and the health care practitioner either independently or together. A number of factors related to the different complementary and/or alternative treatment approaches, stress management techniques, psychotherapeutic techniques and other procedures, techniques, rating scales or diagnostic tools, etc. may be looked at including the following: overall well-being; ease of use; frequency of use; reduction in stress level; reduction in anxiety; reduction in depression; improved energy; level of relaxation, etc.

According to Jonathan C. Smith in his book “Relaxation, Medication, and Mindfulness,” copyright 2005, by Springer Publishing Company, New York, N.Y., the following illnesses are made worse by stress and therefore may specifically respond to optimal stress reduction:

AIDS, any angina, arthritis, asthma, back pain, bradycardia, cancer, cardiac arrhythmia, chemotherapy side effects, chronic fatigue syndrome, colitis, common cold, coronary heart disease, diabetes, fibromyalgia, gastrointestinal disorders, hypertension, hyperventilation, inflammation infection, influenza, injury/healing, insomnia, irritable bowel syndrome, migraine headaches, multiple sclerosis, muscle cramps, myocardial infarction, nausea, peptic ulcer, psoriasis, Raynaud's syndrome, spasmatic dysmenorrhea, tachycardia, and tinnitus.

In addition, Smith's book indicates that the following information may help determine the optimal complementary and/or alternative treatment approaches, stress management techniques, psychotherapeutic techniques and other procedures, techniques, rating scales or diagnostic tools, etc. to be used for a patient:

Sleepiness, disengagement, feeling rested/refreshed, energized, physical relaxation, feeling at ease/peace, Joy/happiness, mental quiet, childlike innocence, thankful/loving, deep mystery, awe/wonder, prayerful, feeling timeless/boundless/infinite/at one, aware, reduced joint and skeletal muscle stress associated with sustained stressful posture and position; reduced fatigue increased energy; reduced general and skeletal muscle tension; relaxed breathing; reduced cognitive autonomic arousal; reduced negative emotion; sustained effortless/passive simple focus.

An inquiry into the effects of the integrated techniques described herein can be used to determine how to program the frequency and duration of the delivery of future complementary and/or alternative (adjunctive) treatment approaches, stress management techniques, psychotherapeutic techniques and other procedures, techniques, rating scales or diagnostic tools, etc. that are delivered with a primary medical intervention or treatment. At subsequent healthcare provider visits further modifications to the program of primary medical treatment and of complementary and/or alternative treatment (adjunctive) approaches, etc. can be made based on further inquiry into the response to complementary and/or alternative treatment approaches, stress management techniques, psychotherapeutic techniques and other procedures, techniques, rating scales or diagnostic tools, etc. that are delivered with a primary medical intervention or treatment. Shared treatment and decision making via giving information to patients, obtaining patient preferences and aligning these to tailor communication to the patient are among the many advantages realized by the present invention.

Reference will now be made in detail to the various aspects of the subject application illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, left, right, above, below and diagonal, are used with respect to the accompanying drawings. Such directional terms used in conjunction with the following description of the drawings should not be construed to limit the scope of the subject application in any manner not explicitly set forth. Additionally, the term “a,” as used in the specification, means “at least one.” The terminology includes the words above specifically mentioned, derivatives thereof, and words of similar import.

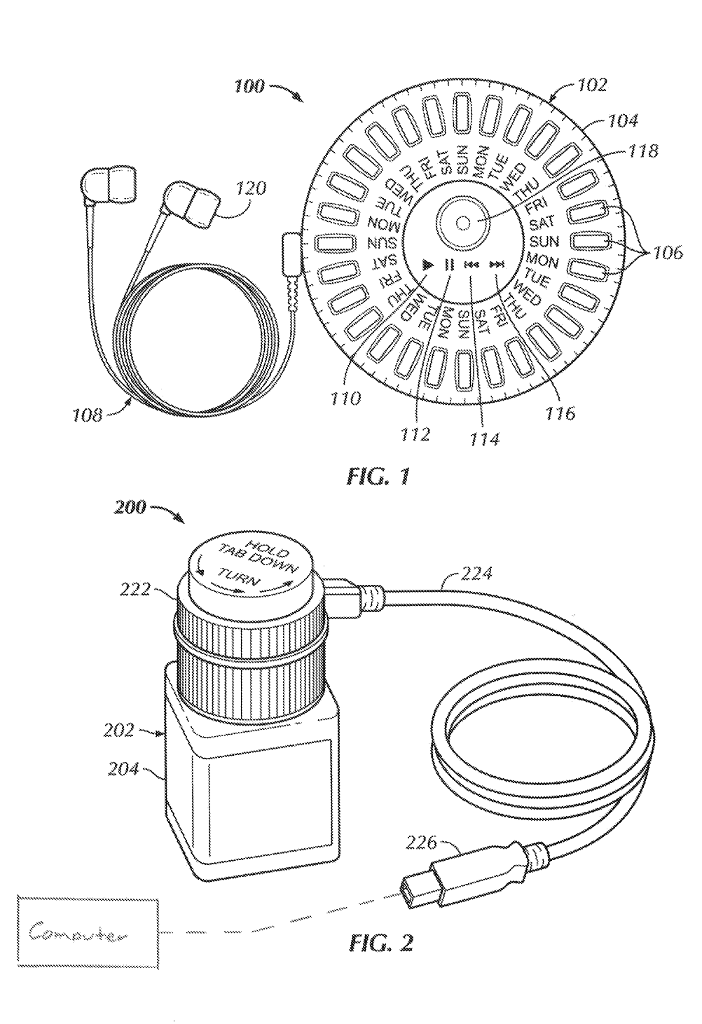

Referring to FIG. 1, there is shown a multimodal medical treatment system 100 constructed in accordance with a first embodiment of the invention. As seen in FIG. 1, the system includes a first delivery system 102 for delivering a primary medical treatment comprising a medication containing device 104. In the illustrated example the medication containing device 104 is a rotatable circular disk containing a plurality of pills 106 similar to a conventional birth control pill dispensing device or other such device from which a single pill is to be dispensed daily. Hence, the primary medical treatment provided by the first delivery system 102 is one of medication provision.

As also shown in FIG. 1, multimodal medical treatment system 100 additionally comprises a second delivery system 108 integrated with the first delivery system 102 for delivering an adjunctive treatment to a patient. In this case, the second delivery system is operable to deliver an analog or digital audio file to the patient. As noted hereinabove, the audio file may be contained on an MP3, a sound card, a chip (e.g., an MP3 digital recording, WMA digital recording, other digital recording, sound card, computer memory or computer chip) or some other way integrated within the second delivery system to record and/or deliver the desired audio file. The second delivery system provides an adjunctive treatment in the form of a recorded message which may take the form of one or more of the many physiological, psychological or other adjunctive treatments enumerated above.

In addition to the mechanism for recording and/or delivering the desired audio file, the second delivery system includes controls for controlling delivery of the audio file to the patient as well as audio delivery/listening devices by which the patient can hear the file. In the illustrated example, the audio file controls include a “play” button 110, a “pause” button 112, a “rewind” button 114 and a “fast-forward” button 116, all of which are in communication with a computer chip or microprocessor which controls the operation of the audio file controls and playing of the audio file through the audio delivery/listening devices. The audio delivery/listening devices by which the patient can hear the file may include at least one speaker 118 incorporated into the body of the medication containing device 104 or earphones 120 that may be plugged into the body of the medication containing device.

Turning to FIG. 2, there is shown a portion of a multimodal medical treatment system 200 constructed in accordance with a further embodiment of the invention. As seen in FIG. 2, system 200 includes a first delivery system 202 for delivering a primary medical treatment comprising a medication containing device 204. In the illustrated example the medication containing device 204 is a medication bottle. The cap 222 of the medication bottle includes an electrical receptacle or port similar to that shown in FIG. 20, discussed below, which receives a first end of a cable 224 that connects the bottle to an unillustrated computer or similar device via connector 226. The cap 222 of the bottle 204 contains circuitry including memory in which is stored adjunctive treatment file(s) whereby the computer or similar device may provide the patient with the adjunctive treatment(s) or instructions therefor upon insertion of connector 226 into the computer or similar device, or the computer or similar device may provide the medication bottle with adjunctive treatment file(s).

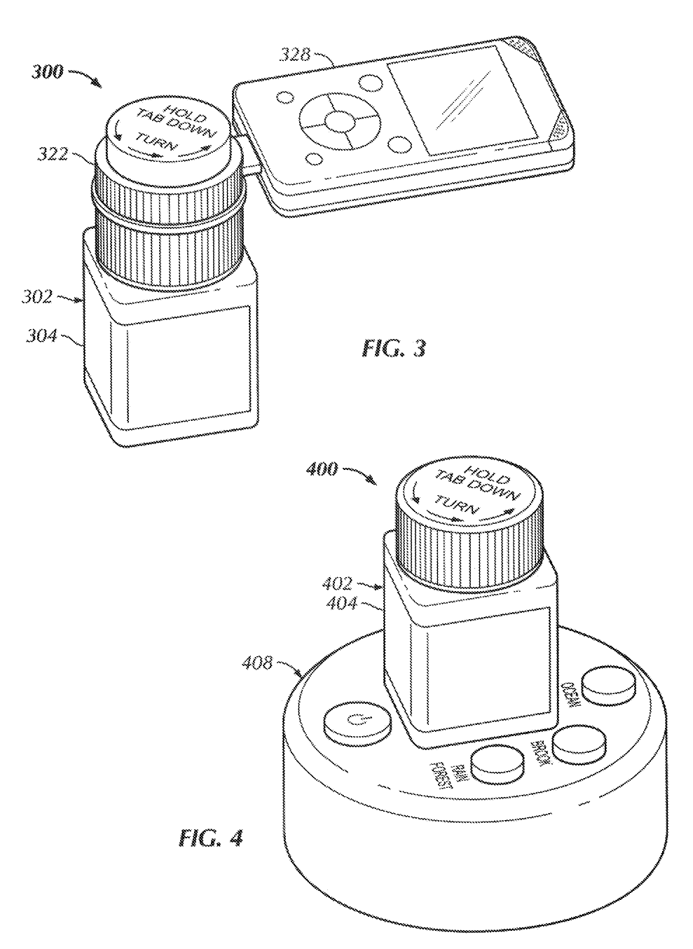

Referring to FIG. 3, there is shown a multimodal medical treatment system 300 constructed in accordance with a further embodiment of the invention. System 300 includes a first delivery system 302 for delivering a primary medical treatment comprising a medication containing device 304 in the form of a medication bottle. The cap 322 of the medication bottle includes an electrical receptacle or port similar to that shown in FIG. 20 which receives an electrical connector that extends from a digital voice recorder 328 which functions as a second delivery system for adjunctive treatment files. According to FIG. 3, the cap 322 of the bottle 304 contains unillustrated circuitry including a microprocessor, a power source and memory in which is stored adjunctive treatment file(s) whereby the digital voice recorder may provide the patient with the adjunctive treatment(s) or instructions therefor upon insertion of the digital voice recorder into the cap. Conversely, the digital voice recorder 328 may be used to program the circuitry contained in the cap 322 to store adjunctive treatment files in the cap circuitry's memory. Alternatively, the audio file may be played through an unillustrated speaker rather than the digital voice recorder.

It will be understood that with respect to the medication containing devices described herein, such devices can either be programmed at the healthcare provider's office, a program can be electronically delivered to the existing container or a new prescription can be programmed by a pharmacist and electronically delivered to the new container. Furthermore, the medication containing devices herein may be programmed with audio, video or audiovisual file of a family member providing instructions/support for performing the adjunctive treatment. In this regard, while the audio, video or audiovisual file may be recorded by a healthcare provider, having a recording in a family member's voice and/or a video recording from a family member supporting the taking of medication and/or an automatic phone call to a family member as a reward for taking medication triggered by opening the medication vial or medication bottle approximates having that family member in the house and may be especially comforting to the patient.

FIG. 4 is a perspective view of a medication bottle coupled with a sound producing device for enabling a user to receive an audio message concerning an adjunctive treatment. In particular, FIG. 4 shows a multimodal medical treatment system 400 comprising a first delivery system 402 for delivering a primary medical treatment in the form a medication containing device or bottle 404. In the example shown in FIG. 4, the second delivery system 408 for delivering the adjunctive treatment is a sound producing device which may selectively simulate the soothing sounds of moving water such as those produced by an ocean, a rain forest or a brook.

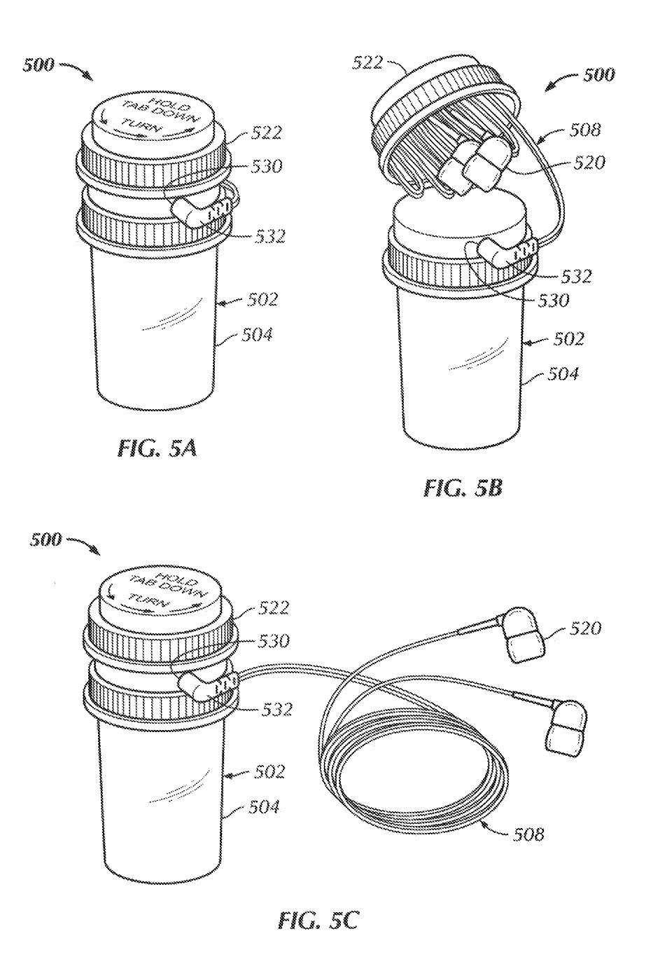

FIGS. 5A-5C disclose in sequence the method for implementing a multimodal medical treatment system 500. System 500 comprises a first delivery system 502 for delivering a primary medical treatment in the form of a medication containing device 504. In the illustrated example the medication containing device 504 is a medication bottle. System 500 further includes a second delivery system for delivering an adjunctive treatment. In particular, the second delivery system is incorporated into the cap 522 of the medication bottle. That is, the cap 522 includes an electrical receptacle or port 530 into which is inserted an electrical connector or plug 532 of a set of earphones 520 that is housed within the cap 522 when not in use. The cap 522 of the bottle 504 contains circuitry including memory in which is stored audio adjunctive treatment file(s) that the patient can listen to through earphones 520.

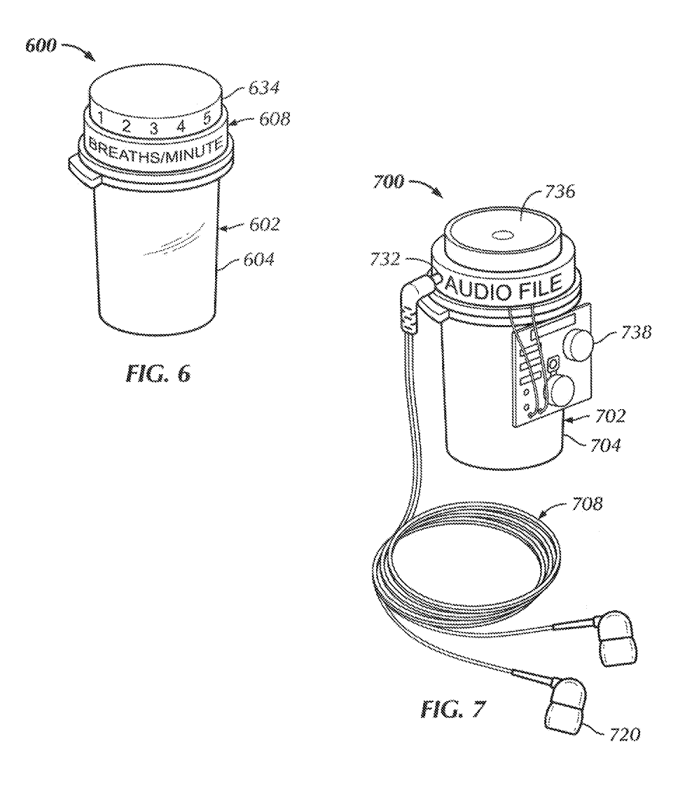

FIG. 6 shows a multimodal medical treatment system 600 comprising a first delivery system 602 for delivering a primary medical treatment in the form a medication containing device or bottle 604. In the example shown in FIG. 6, the second delivery system 608 for delivering the adjunctive treatment is a breathing rate controlling tool in the form of an adjustable dial 634 for indicating to a patient the breaths per minute the patient should take pursuant to a breathing regimen prescribed by the patient's healthcare provider.

FIG. 7 shows a multimodal medical treatment system 700 comprising a first delivery system 702 for delivering a primary medical treatment in the form a medication containing device or bottle 704. Similar to earlier described systems, system 700 delivers an audio adjunctive treatment file through earphones 720 which are connected to cap 722 via plug or connector 732. FIG. 7 also shows the top of the bottle 704 removed to expose the electrical circuitry, including memory 736 and a combined central processing unit and power source 738 via which the audio file is processed, stored and delivered to the patient.

FIG. 8 shows a multimodal treatment system 800 similar in construction to the system shown in FIG. 7. However, the top 840 of the bottle is shown on top of cap 822 and covering certain components of the electrical circuitry. Further, the cap 822 of the bottle 804 indicates that the adjunctive audio file delivered by the system is directed to stress relief therapy.

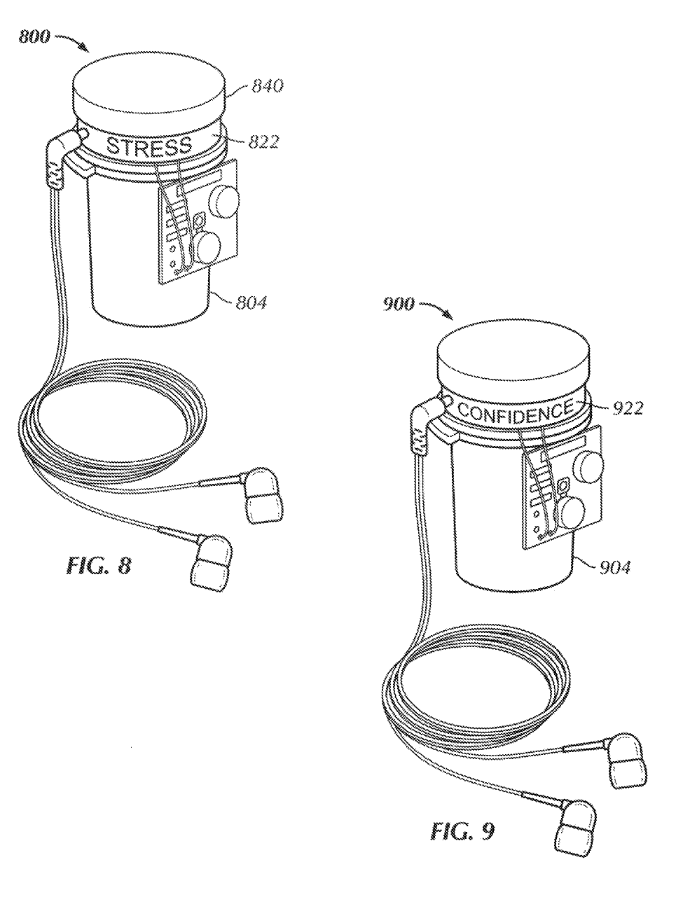

FIG. 9 shows a multimodal treatment system 900 similar in construction to the systems shown in FIGS. 7 and 8. However, the cap 922 of the bottle 904 indicates that the adjunctive audio file delivered by the system is directed to confidence building therapy.

FIG. 10 shows a multimodal treatment system 1000 similar in construction to the systems shown in FIGS. 7, 8 and 9. However, the cap 1022 of the bottle 1004 indicates that the adjunctive audio file delivered by the system is directed to relaxation therapy. In addition, the top 1040 of the bottle indicates that the system can deliver multiple audio files as indicated by selections 1, 2, 3, 4 and 5.

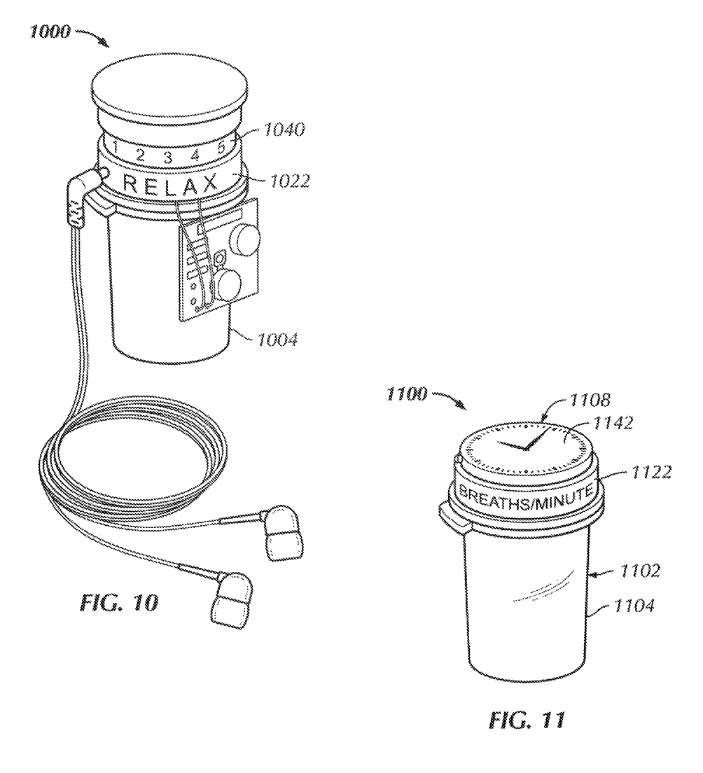

FIG. 11 shows a multimodal treatment system 1100 including a first delivery system 1102 for delivering a primary medical treatment comprising a medication containing device 1104 in the form of a medication bottle. The cap 1122 of the medication bottle includes a second delivery system 1108 for delivering the adjunctive treatment. As shown in FIG. 11, the second delivery system 1108 includes a breathing rate controlling tool in the form of a timepiece or other information device 1142 integrated within the cap 1122 for providing a readily available means for timing of the number of breaths the patient should take permit or other information pursuant to a breathing regimen or other adjunctive treatment prescribed by the patient's healthcare provider.

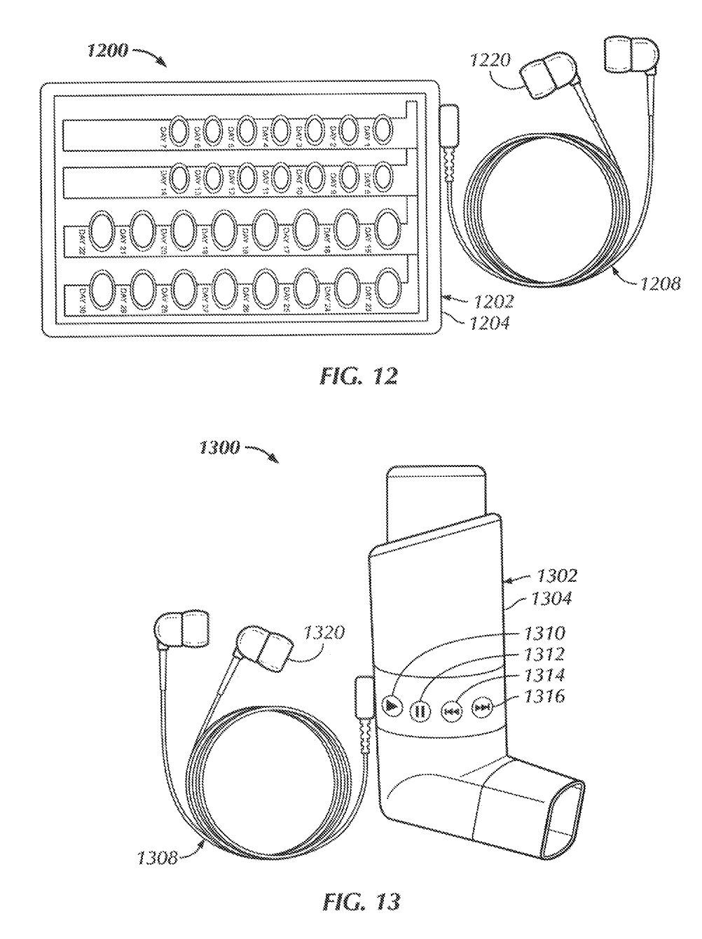

FIG. 12 shows a multimodal treatment system 1200 including a first delivery system 1202 for delivering a primary medical treatment comprising a medication containing device 1204 in the form of a starter titration package for new medication to be taken by the patient. Multimodal medical treatment system 1200 additionally comprises a second delivery system 1208 incorporated into the first delivery system 1202 for delivering an adjunctive treatment to a patient. In this case, the second delivery system is operable to deliver an analog or digital audio file to the patient via earphones 1220 that may be plugged into the body of the medication containing device 1204.

FIG. 13 shows a multimodal treatment system 1300 including a first delivery system 1302 for delivering a primary medical treatment comprising a medication containing device 1304 in the form of an inhaler. Multimodal medical treatment system 1300 additionally comprises a second delivery system 1308 incorporated into the first delivery system 1302 for delivering an adjunctive treatment to a patient. In this case, the second delivery system is operable to deliver an analog or digital audio file to the patient via earphones 1320 that may be plugged into the body of the medication containing device 1304.

Similar to FIG. 1, the second delivery system includes controls for controlling delivery of the audio file to the patient. In the illustrated example, the audio file controls include a “play” button 1310, a “pause” button 1312, a “rewind” button 1314 and a “fast-forward” button 1316.

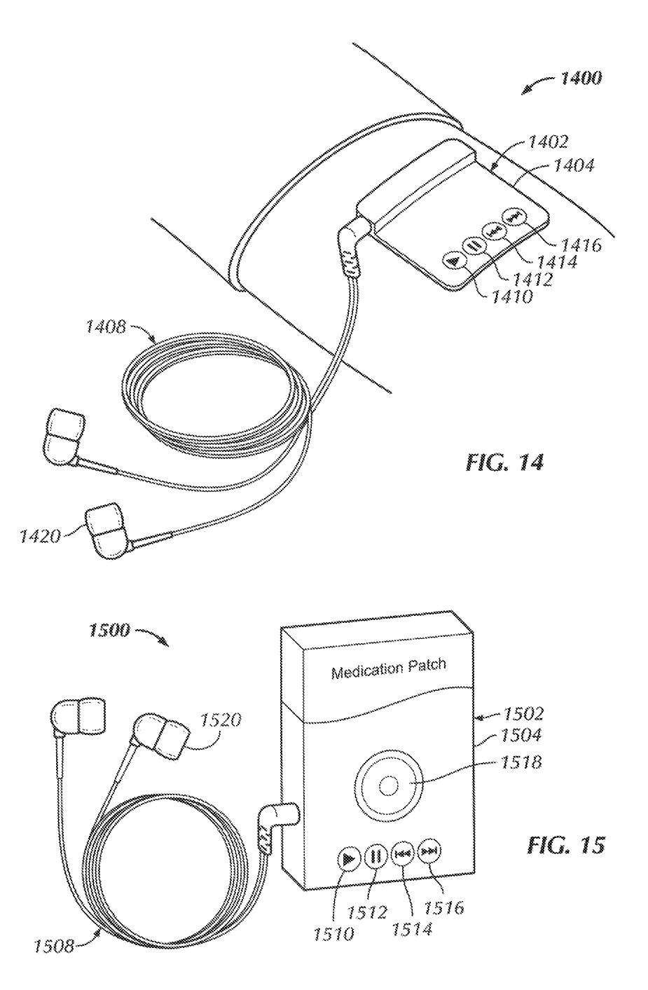

FIG. 14 shows a multimodal treatment system 1400 including a first delivery system 1402 for delivering a primary medical treatment comprising a medication containing device 1404 in the form of medicated patch. Multimodal medical treatment system 1400 additionally comprises a second delivery system 1408 incorporated into the first delivery system 1402 for delivering an adjunctive treatment to a patient. In this case, the second delivery system is operable to deliver an analog or digital audio file to the patient via earphones 1420 that may be plugged into the body of the medication containing device 1404.

The second delivery system includes controls for controlling delivery of the audio file to the patient. In the illustrated example, the audio file controls include a “play” button 1410, a “pause” button 1412, a “rewind” button 1414 and a “fast-forward” button 1416.

FIG. 15 shows a multimodal treatment system 1500 including a first delivery system 1502 for delivering a primary medical treatment comprising a medication containing device 1504 in the form of a box or other container which contains medicated patches. Multimodal medical treatment system 1500 additionally comprises a second delivery system 1508 incorporated into the first delivery system 1502 for delivering an adjunctive treatment to a patient. In this case, the second delivery system is operable to deliver an analog or digital audio file to the patient via earphones 1520 that may be plugged into the body of the medication containing device 1504 or via a speaker 1518 incorporated into box 1504.

The second delivery system includes controls for controlling delivery of the audio file to the patient. In the illustrated example, the audio file controls include a “play” button 1510, a “pause” button 1512, a “rewind” button 1514 and a “fast-forward” button 1516.

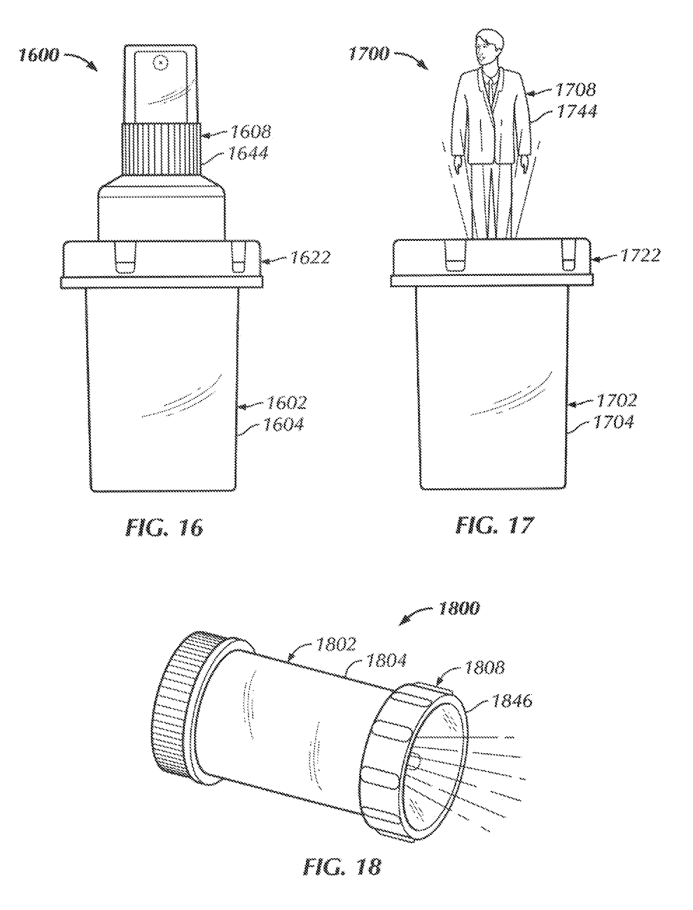

FIG. 16 shows a multimodal treatment system 1600 including a first delivery system 1602 for delivering a primary medical treatment comprising a medication containing device 1604 in the form of a medication bottle. The cap 1622 of the medication bottle includes a second delivery system 1608 for delivering the adjunctive treatment. As shown in FIG. 16, the second delivery system 1608 includes an aerosol dispenser 1644 integrated within the cap 1622 for providing a spray of scented liquid pursuant to an aroma therapy regimen prescribed by the patient's healthcare provider.

FIG. 17 shows a multimodal treatment system 1700 including a first delivery system 1702 for delivering a primary medical treatment comprising a medication containing device 1704 in the form of a medication bottle. The cap 1722 of the medication bottle includes a second delivery system 1708 for delivering the adjunctive treatment. As shown in FIG. 17, the second delivery system 1708 includes a holographic image projector integrated within the cap 1722 for projecting a holographic image 1744 and an unillustrated speaker for providing the patient with a combined audio and video file prescribed by the patient's healthcare provider.

FIG. 18 shows a multimodal treatment system 1800 including a first delivery system 1802 for delivering a primary medical treatment comprising a medication containing device 1804 in the form of a medication bottle. The base of the medication bottle includes a second delivery system 1808 for delivering the adjunctive treatment. As shown in FIG. 18, the second delivery system 1808 includes a light projector 1846 for providing light pursuant to a light therapy regimen prescribed by the patient's healthcare provider.

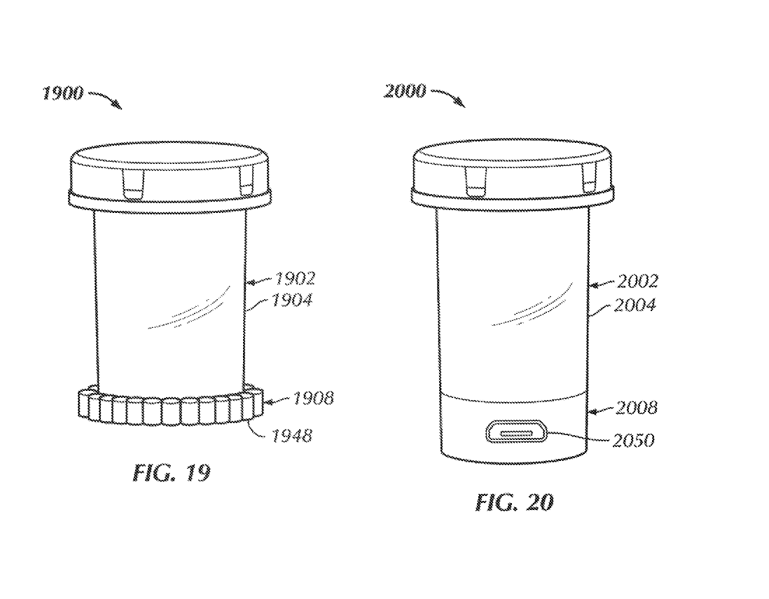

FIG. 19 shows a multimodal treatment system 1900 including a first delivery system 1902 for delivering a primary medical treatment comprising a medication containing device 1904 in the form of a medication bottle. The base of the medication bottle includes a second delivery system 1908 for delivering the adjunctive treatment. As shown in FIG. 19, the second delivery system 1908 includes one or more permanent magnets 1948 for providing magnetism pursuant to a magnetic therapy regimen prescribed by the patient's healthcare provider.

FIG. 20 shows a multimodal treatment system 2000 including a first delivery system 2002 for delivering a primary medical treatment comprising a medication containing device 2004 in the form of a medication bottle. The base of the medication bottle includes a second delivery system 2008 for delivering the adjunctive treatment. As shown in FIG. 20, the second delivery system 2008 includes an electrical port 2050 in the base of the medication bottle for enabling connection to a device for delivering an adjunctive treatment. More particularly, electrical port 2050 may be a USB or similar port which is in communication with unillustrated circuitry housed within the base of the medication bottle in which is programmed an audio, video or audiovisual file embodying the adjunctive treatment. When a suitable cable is inserted into the electrical port and an unillustrated computer or similar device the adjunctive treatment file may be executed on the computer or similar device whereby the user may experience the adjunctive treatment.

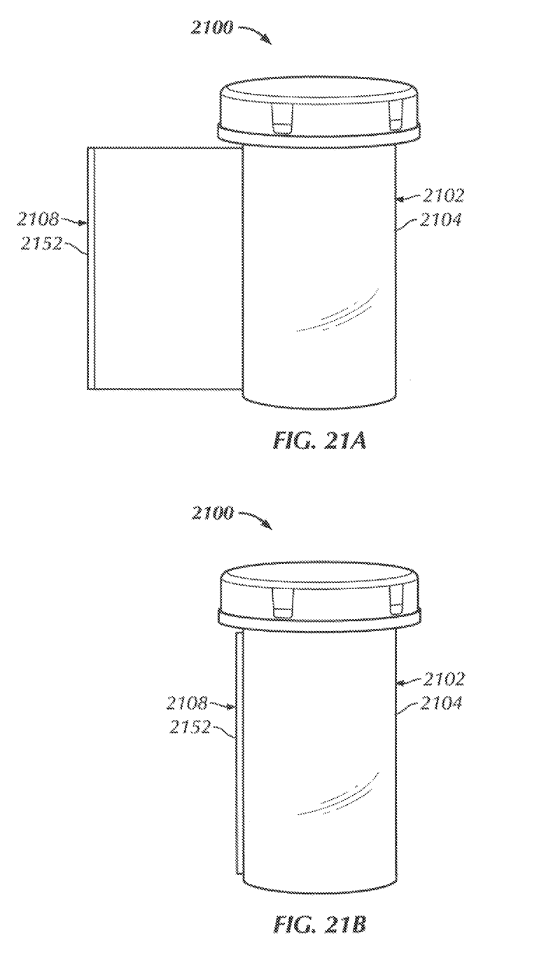

FIGS. 21A and 21B shows a multimodal treatment system 2100 including a first delivery system 2102 for delivering a primary medical treatment comprising a medication containing device 2104 in the form of a medication bottle. The bottle 2104 carries on its side a second delivery system 2008 for delivering the adjunctive treatment. In the illustrated example, the second delivery system comprises a flexible electronic screen 2152, such as a flexible LED or LCD display screen, that may be extended from the bottle as shown in FIG. 21A to display a visual message to the patient containing the prescribed adjunctive treatment. After use, the screen 2152 may be wrapped around or retracted into the bottle as shown in FIG. 21B.

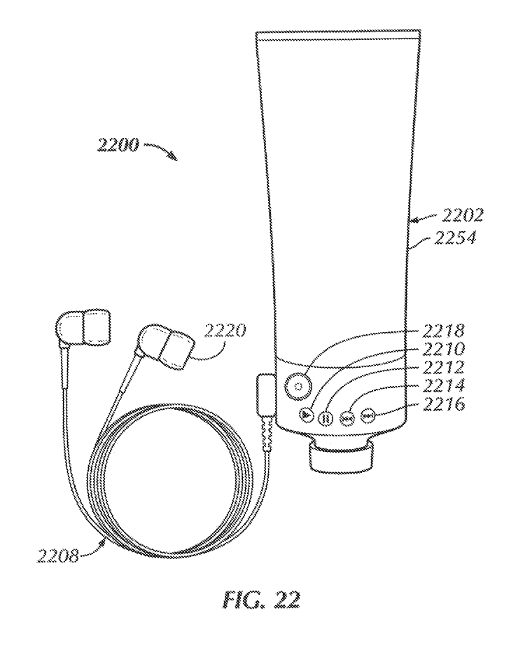

FIG. 22 shows a multimodal treatment system 2200 including a first delivery system 2202 for delivering a primary medical treatment comprising a medication containing device 2204 in the form of a squeezable medication tube. Multimodal medical treatment system 2200 additionally comprises a second delivery system 2208 incorporated into the first delivery system 2202 for delivering an adjunctive treatment to a patient. In this case, the second delivery system is operable to deliver an analog or digital audio file to the patient via earphones 2220 that may be plugged into the body of the medication containing device 2204 or via a speaker 2218 incorporated into the body of device 2204.

The second delivery system includes controls for controlling delivery of the audio file to the patient. In the illustrated example, the audio file controls include a “play” button 2210, a “pause” button 2212, a “rewind” button 2214 and a “fast-forward” button 2216.

The following are additional design variants that are contemplated to be within the scope and spirit of the present invention.

A. The cap of the medication bottle will contain all electronics, including a speaker. When the cap is removed or touched, the audio file that has been downloaded into the cap's memory is played through the speaker in the cap so that the patient can hear the alternative/complementary treatment or protocol. The audio file could be downloaded in the following ways: pharmacist loads the file onto the device according to a script, manually or otherwise; bottle caps with self-load once unscrewed; patient could download programs at home; caps may be pre-loaded.

B. The cap of the medication bottle will contain all electronics. When the cap is removed or touched, it will send a signal, of the type to be determined, which will trigger a smartphone app to play the audio file on the cell phone. The file will have already been downloaded to the cell phone at a previous time or perhaps the first time the bottle is opened it will send a signal to the cell phone to grab the file from the internet. Every subsequent use will automatically recheck to make sure the correct file is still on the cell phone, and be able to download the file again if it is missing from the cell phone. If the correct file is still on the smart phone, a smart phone application is triggered to play the audio file on the phone.

C. The cap of the medication bottle will contain all electronics. When the cap is removed it will send a signal, of the type to be determined, which will trigger a smartphone app to play the audio file from the cell phone. The file will have already been download to the cell phone at a previous time or perhaps the first time the bottle is opened it will send a signal to the cell phone to grab the file. Every subsequent use will recheck to make sure the correct file is still on the cell phone, and be able to download the file again if it is missing from the cell phone. The cell phone will, in turn, send the message to a blue tooth equipped device accessible by the user so that the adjunctive treatment file could be heard in private.

D. The combination of A, B or C above where the patient would have the option of listening on earbuds (which may be shipped with the bottle) that may be plugged into a headphone port on the side of the cap, listening from a speaker built into the cap, listening on the cell phone, or listening on a wireless connection from the cell phone when the pill bottle is opened.

E. The cap of the medication bottle will contain the circuit board containing all electronic components and the speaker. The cap can convert to a stand-alone over-the-ear audio player and the audio will play out of this speaker when the cap is either on the bottle or removed from bottle. The cap will have an attachment that will hook over the outer ear allowing the cap to be positioned over the external auditory canal for optimal sound quality.

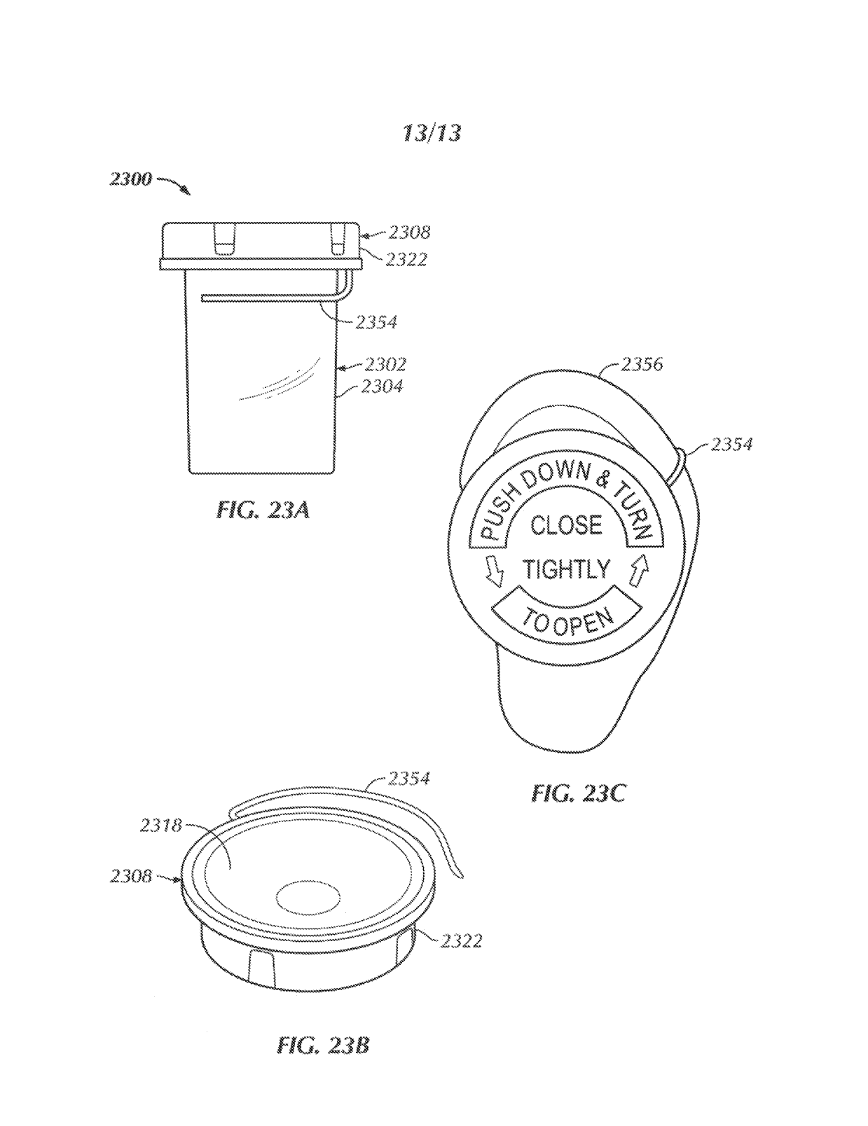

FIGS. 23A-23C reflect the construction and operation of variant E described immediately hereabove. FIG. 23A shows a multimodal treatment system 2200 including a first delivery system 2302 for delivering a primary medical treatment comprising a medication containing device 2304 in the form of medication bottle. Multimodal medical treatment system 2300 additionally comprises a second delivery system 2308 incorporated into the first delivery system 2302 for delivering an adjunctive treatment to a patient. In this case, the second delivery system is a Bluetooth enabled communications device provided in cap 2322 which is operable to deliver an analog or digital audio file to the patient. The Bluetooth enabled communications device includes a suspension wire or similar device 2354 which may or may not function as a Bluetooth antenna that may be suspended over the ear 2356 of a patient in the manner shown in FIG. 23C.

Referring to FIG. 23B, there is shown a speaker 2318 provided in the interior of the cap 2322 which faces the patient's ear to deliver the adjunctive treatment when the cap is donned by the patient in the manner shown in FIG. 23C. The cap 2322 contains all of the necessary electronics to effectuate a Bluetooth connection to a remote broadcast source. In the alternative, the cap 2322, speaker 2318 and its incorporated circuitry may be a stand-alone device for delivering a pre-recorded adjunctive treatment message contained within the cap.

It will be appreciated by those skilled in the art that changes could be made to the embodiments described above without departing from the broad inventive concept thereof. For example, additional components and steps can be added to the various syringe cartridge systems. It is to be understood, therefore, that this invention is not limited to the particular embodiment disclosed, but it is intended to cover modifications within the spirit and scope of the present invention as defined by the appended claims.

I claim:

1. A system for multimodal medical treatment comprising:
a first delivery system for delivering a primary medical treatment to a patient; and
a second delivery system integrated with the first delivery system, the second delivery system including:
a memory having an audio file, a video file or an audiovisual file of an adjunctive medical treatment, and
an output delivery device operatively connected to the memory for delivering the adjunctive medical treatment to the patient.

2. The system of claim 1, wherein the first delivery system comprises a medication containing device.

3. The system of claim 2, wherein the medication containing device is a patch.

4. The system of claim 2, wherein the medication containing device is an inhaler.

5. The system of claim 1, wherein the output delivery device is a screen, earphones, a speaker, or a holographic projection.

6. The system of claim 1, wherein the output delivery device executes the audio file, the video file or the audiovisual file.

7. A method for multimodal medical treatment optimization comprising:
providing a patient with the multimodal medical treatment optimization system of claim 1;
delivering the primary medical treatment via the first delivery system;
delivering the adjunctive medical treatment via the second delivery system; and
adjusting at least one of the primary medical treatment and the adjunctive medical treatment as a result of the patient's physiological or psychological response to at least one of the primary medical treatment and the adjunctive medical treatment.

8. A system for multimodal medical treatment comprising:
a first delivery system for delivering a primary medical treatment to a patient; and
a second delivery system integrated with the first delivery system, the second delivery system including an adjunctive medical treatment comprising at least one of light therapy, aromatherapy and magnetic therapy.

9. The system of claim 6, wherein the first delivery system comprises a medication containing device.

10. The system of claim 6, wherein the second delivery system comprises an aerosol dispensing device attached to the first delivery system.

11. The system of claim 6, wherein the second delivery system comprises a light illuminating device attached to the first delivery system.

12. The system of claim 6, wherein the second delivery system comprises a magnet attached to the first delivery system.

13. A multimodal medical treatment system comprising:
a medication containing device for housing a primary medical treatment; and
an audiovisual module having stored audio, video or audiovisual files of an adjunctive medical treatment, and
wherein when the medication containing device is activated, playback of the audio, video or audiovisual files begins.

14. The system of claim 13, wherein the audiovisual module includes an output delivery device.

15. The system of claim 14, wherein the output delivery device is a screen, earphones, a speaker, a USB port or a holographic projection.

16. The system of claim 15, wherein the screen is an LED or LCD screen.

17. The system of claim 15, wherein the screen is extendable and retractable.

18. A multimodal medical treatment system comprising:
a medication containing device for housing a primary medical treatment, wherein the medication containing device includes an integral breathing rate controlling tool.

19. The system of claim 18, wherein the tool comprises a timepiece

20. The system of claim 18, wherein the tool comprises an adjustable dial.
